(19) United States
(12) Patent Application Publication
(10) Pub. No.: US 2023/0303622 A1
(43) Pub. Date: Sep. 28, 2023

(54) MODELING CONDITIONS FOR TANGENTIAL FLOW FILTRATION PROCESSES FOR PROTEIN PURIFICATION

(71) Applicant: Ohio State Innovation Foundation, Columbus, OH (US)

(72) Inventors: Ivan SUSIN PIRES, Cambridge, MA (US); Andre PALMER, Bexley, OH (US)

(21) Appl. No.: 18/023,950

(22) PCT Filed: Aug. 30, 2021
(86) PCT No.: PCT/US21/48241

§ 371 Date: Feb. 28, 2023

Related U.S. Application Data
(60) Provisional application No. 63/071,555, filed on Aug. 28, 2020.

(51) Int. Cl. C07K 1/34; C07K 1/16

(52) U.S. Cl. CPC C07K 1/34; C07K 1/16

(57) ABSTRACT

Tangential flow filtration (TFF) is a size-based separation method conventionally used for buffer exchange, concentration, pathogen removal, and for coarse purification. Disclosed herein, TFF was used for selective purification of proteins in their complexed form. In some examples, this process was demonstrated to recover human serum albumin (HSA) in its complexed form from an artificially produced mixture of hemoglobin (Hb) and HSA and from plasma using an anti-HSA polyclonal immunoglobulin G (IgG) as the target-protein binding molecule (TPBM). Moreover, another embodiment of the method recovered haptoglobin (Hp) in its complexed form from human Cohn Fraction IV using Hb as the TPBM. In addition, a mathematical model used to describe the TFF purification process provided that product recovery could be increased without loss of purity by introducing TFF filters with the same MWCO in series. The following disclosure presents a new method for selective purification of proteins using TFF and a simple mathematical model to describe and predict the performance of TFF systems.

### CROSS-REFERENCE TO RELATED APPLICATIONS

This application claims benefit of U.S. Provisional Application No. 63/071,555, filed Aug. 28, 2020, which is hereby incorporated herein by reference in its entirety.

### BACKGROUND

Protein purification is the foundation for most of the biopharmaceuticals in the marketplace. The purified protein product can be used in all aspects of healthcare for applications such as therapeutics, diagnostic agents, and research. Therapeutic proteins serve to supplement or restore biological function (such as plasma components) or as targeted therapies (such as monoclonal antibodies). Diagnostic proteins primarily include antibody detection systems and other bioassays used to detect markers of disease or biological dysfunction. Finally, given that research is required to validate protein usage, research proteins encompass all application areas. Moreover, in addition to the biopharmaceutical market, recent industrial use of proteins as biocatalysts has further increased the demand for proteins. Thus, given the high demand for proteins, many protein purification methods have been developed over the years. Unfortunately, although these new methods have reduced the overall cost of proteins, a substantial fraction of the manufacturing cost of proteins still comes from the protein purification process.

Most large-scale processes for protein purification rely on several chromatography steps to achieve the desired protein purity. These chromatography steps must each be individually optimized for the specific protein of interest. Moreover, conventional column chromatography techniques are associated with high production costs and low volumetric throughput. Thus, there have been many research efforts to develop non-chromatographic purification techniques such as protein precipitation or liquid-liquid separation. Interestingly, membrane-based size exclusion separation methods have not been widely used for selective protein purification. Current uses of membrane filtration mainly include solution clarification (microfiltration), virus and bacterial removal, protein concentration, and buffer exchange. More recent membrane separation methods have started to use both size and charge for separation (termed high-performance tangential flow filtration). However, the use of membrane-based separation for selective protein purification is still not widely used in industry.

### SUMMARY

Described herein are methods which employ ultrafiltration (e.g., tangential flow filtration (TFF)) to purify a target protein (TP) from a mixture of proteins by exploiting molecular size changes that arise from the formation of a protein-protein complex consisting of a TP and a TP binding molecule (TPBM). Previous studies have used an approach referred to as “affinity filtration”. However, most of the studies used solid substrates that adsorbed the TP or had ligands covalently attached to the substrate that were specific to the TP. Later studies employed water soluble polymers bound to ligands as the TPBM. Yet, in all these systems, there was a requirement of either an insoluble affinity matrix or high molecular weight (MW) polymer conjugated to a ligand to facilitate selective binding of the TP. The disclosed method demonstrates that the range of applicable TPBM is wider, and can consist of simple proteins capable of selectively complexing with the TP. Proteins are viable alternatives to polymers to be used as TPBM, since polymers may adsorb to filter membranes, thus increasing membrane fouling, and polymeric solutions tend to have high viscosity which can decrease the flux through the membrane. Moreover, proteins may be engineered to have a desired MW and affinity for increased performance in the proposed system.

Briefly, the methods described herein employ ultrafiltration with a defined MW cut off (MWCO) membrane to first permeate the TP and other impurities that are below the MWCO of the membrane, as well as set the maximum size/MW of the protein species in the filtrate. In some embodiments, a TPBM may then be added to the filtrate to selectively create a protein-protein complex with the TP in the protein mixture that is above the MWCO of the original membrane. With only the TP-TPBM complex in the protein mixture above the MWCO of the original membrane, the TP-TPBM can be selectively separated from the other low MW protein components and impurities in the filtrate by passing it through the original MWCO TFF membrane. These methods are schematically illustrated, for example, in FIG. 1.

Also provided are methods for efficiently separating a target species from a solution containing one or more impurities using ultrafiltration. These methods can incorporate mathematical modeling steps which can be used to optimize process conditions (e.g., number of diafiltration volumes, the selection of appropriate filtration membranes, or any combination thereof) to afford, for example, a desired degree of target species purity. For example, provided are methods for separating a target species from a solution containing one or more impurities, the method comprising: (i) estimating a retention factor (e.g., fraction of an individual species retained on the filter membrane) of a target species from the molecular weight of the target species and a retention curve of a filter membrane; (ii) calculating a number of diafiltration volumes needed to afford a desired fraction of target species based on the estimated retention factor for the target species on the filter membrane and the net individual species molar flowrate of the target species into the system; and (iii) filtering the solution by ultrafiltration against the filtration membrane having the retention curve from (i) and using the number of diafiltration volumes from (ii), thereby forming a fraction substantially comprising the additional species and another fraction substantially comprising the target species. These mathematical modeling steps can likewise be used to describe and predict product recovery and purity, and/or to increase selective recovery of the target species (e.g., improve product purity).

### BRIEF DESCRIPTION OF THE DRAWINGS

FIG. 1 shows an illustration of affinity ultrafiltration facilitated via protein-protein interactions. Starting with a mixture of proteins/particulates (1) (example: cell lysate, human plasma, etc.), the mixture is filtered through a membrane with an appropriate MWCO that permeates the TP along with low MW impurities (2). Then a TPBM (i.e. antibody or equivalent, etc.) specific to the TP is introduced into the filtrate, forming a TP-TPBM protein complex that is larger than the MWCO of the original membrane (3). The solution with the newly formed TP-TPBM protein complex is then refiltered through the same MWCO membrane leading to retention of the isolated TP-TPBM protein complex of interest and removal of low MW impurities (4). The isolated TP-TPBM protein complex can then be dissociated to yield free TP and TPBM via appropriate buffer exchange under conditions that would facilitate their dissociation (5). With the individual species (TP and TPBM) dissociated in solution, the TP can be separated from the TPBM using a MWCO membrane that is between the MW of the TP and TPBM (6). Note: both the TPBM in the retentate and TP in the filtrate can be buffer exchanged via TFF into appropriate buffers to remove the dissociating agent and concentrate the separated TP and TPBM.

FIG. 2 shows a general production scheme for the purification of the Hb-Hp protein complex from Cohn fraction IV paste using TFF.

FIG. 3 shows a diagram showing the dissociation of Hb from the Hb-Hp complex to isolate Hp. Numbers in brackets indicate the number of diafiltration volumes.

FIG. 4A-FIG. 4B show a diagram of the single stage TFF system (indicated by the gray dashed lines) used for modeling the TFF process (4A). Manufacturer's specifications (specs) for 30, 50, 70 and 100 kDa mPES HF filters (arrows indicate specification of more than or less than) and the fitted retention curves based on the Hill equation for each HF filter (4B).

FIG. 5A-FIG. 5B show HPLC-SEC of mixtures of HSA and IgG. (5A) Full chromatogram with pure species. (5B) Change in absorbance based on the difference between the mixture chromatogram and the pure species chromatograms. Quotation marks were used, since the difference in spectra is not a perfect description of the bound species. The HSA concentration was set to 0.08 mg/mL and approximate IgG concentrations of 0.04 mg/mL, 0.16 mg/mL and 0.4 mg/mL were employed. Numbers in parenthesis indicate the approximate mass ratio. *corresponds to the change in absorbance when only considering IgG as an initial species.

FIG. 6A-FIG. 6C show the purification of HSA-IgG complex from artificially produced HSA and Hb mixture. (6A) HPLC-SEC of HSA, Hb and mixture of HSA and Hb. (6B) HPLC-SEC of first and last permeates of 70 kDa HF filter. (6C) HPLC-SEC of initial HSA and Hb mixture before and after IgG addition and isolated HSA-IgG complex. Normalization accounted for the different total volume of samples.

FIG. 7 shows the purification of HSA-IgG complex from plasma. 70P-50R represents the fraction of plasma that permeated through the 70 kDa HF filter and was retained on a 50 kDa HF filter. Normalization was used to account for different total volumes of samples.

FIG. 8 shows an illustration of using the protein complex affinity purification method to isolate Hp from a complex mixture (the complex mixture consisted of Cohn Fraction IV and the dissociating agent was urea).

FIG. 9A-FIG. 9J show a comparison of the hypothetical and experimentally measured HPLC-SEC elution chromatogram at various stages of processing to purify the Hb-Hp complex.

FIG. 10 shows the SDS-PAGE of the purified Hb-Hp complex and mixture of Hp and Hp-Hb obtained from dissociation and separation of Hb from the purified Hb-Hp complex. Lane 1: Isolated Hb-Hp complex. Lane 2: Mixture of Hp and Hb-Hp. Lane 3: 100 kDa permeate. Lane 4: 100 kDa permeate with added Hb. Abbreviations: transferrin (Tf); haptoglobin (Hp), human serum albumin (HSA), hemoglobin (Hb), beta chain of haptoglobin (β Hp), alpha chain of haptoglobin (α Hp).

FIG. 11A-FIG. 11E show model results for the experiments performed earlier in this study. (11A) Estimated retention curves and the expected retention for the species used in these studies. (11B) Separation of HSA and low MW species from large MW impurities using a 70 kDa HF filter. (11C) Separation of HSA-IgG complexes from low MW species using a 70 kDa HF filter. (11D) Separation of Hp (tetramers and higher order Hp species, as well as trimers indicated by the dotted line) and low MW species from large MW impurities using a 100 kDa HF filter. (11E) Separation of Hp-Hb complexes (tetramers and higher order Hp species, as well as trimers indicated by the dotted line) from low MW impurities using a 100 kDa HF filter. Shading indicates the range of curves that could comprise the permeate (P) and retentate (R).

FIG. 12A-FIG. 12B show model results with association and dissociation reactions included for the initial HSA-IgG recovery from artificial HSA and Hb mixture experiment. (12A) Concentration profile of species (IgG, HSA, Hb and IgG-HSA complex) assuming a single IgG species. (12B) Semi-log plot of species concentration assuming a single IgG species. The model parameters used included a reference initial concentration of 3*10⁻⁷ M (C₀), time to complete a diafiltration volume (τ) of one hour, dissociation constant (K_{D}) of 10⁻⁸ M and rate of association constant of 10⁶ M⁻¹ s⁻¹ (value obtained from the literature 1411). The initial normalized concentrations used were 2, 1 and 2 for HSA, IgG and Hb, respectively. The HSA-IgG complex was assumed to have a MW of 220 kDa, in order to determine its retention on the TFF filtration system.

FIG. 13A-FIG. 13E show model results using individual TFF modules staged in series. (13A) Diagram of TFF modules staged in series. (13B) Serial staging of TFF modules for HSA-IgG recovery. (13C) Serial staging of TFF modules for Hb-Hp complex recovery. The retained species are at or above the curves for the complexes, while the permeated species are at or below the curves for the low MW impurities. (13D) Trade-off between retention of the HSA-IgG complex and removal of impurities using one, two, or three staged TFF systems (distance between each circle corresponded to 1 diafiltration volume). (13E) Trade-off between retention of the Hb-Hp complex and removal of impurities using one, two, or three staged TFF systems (distance between each circle corresponded to 10 diafiltration volumes).

FIG. 14A-FIG. 14D show a comparison of the TFF separation model with chemical reactions to the TFF separation model with no chemical reactions. (14A) Concentration profile of species assuming no chemical reactions. (14B) Semi-log plot of concentration profile of species assuming no chemical reactions. (14C) Concentration profile of species including chemical reaction terms. (14D) Semi-log plot of concentration profile of species including chemical reaction terms. Model parameters: reference initial concentration of 10⁻⁵ M (C₀), time for a complete diafiltration volume (τ) of one-tenth of an hour, dissociation constant (K_{D}) of 10⁻¹² M and rate of association constant of 10⁶ M⁻¹ s⁻¹.

### DETAILED DESCRIPTION

### Definitions

As used herein, the term “tangential-flow filtration” refers to a process in which the fluid mixture containing the components to be separated by filtration is recirculated at high velocities tangential to the plane of the filtration membrane to reduce fouling of the filter. In such filtrations a pressure differential is applied along the length of the filtration membrane to cause the fluid and filterable solutes to flow through the membrane (i.e. filter). This filtration is suitably conducted as a batch process as well as a continuous-flow process. For example, the solution may be passed repeatedly over the membrane while that fluid which passes through the filter is continually drawn off into a separate unit or the solution is passed once over the membrane and the fluid passing through the filter is processed (e.g., continually processed) downstream.

As used herein, the term “ultrafiltration” is used for processes employing membranes rated for retaining solutes having a molecular weight between about 1 kDa and 1000 kDa.

As used herein, the term “reverse osmosis” refers to processes employing membranes capable of retaining solutes of a molecular weight less than 1 kDa such as salts and other low molecular weight solutes.

As used herein, the term “microfiltration” refers to processes employing membranes in the 0.1 to 10 micron pore size range.

As used herein, the expression “transmembrane pressure” or “TMP” refers to the pressure differential gradient that is applied along the length of a filtration membrane to cause fluid and filterable solutes to flow through the filter.

The term “hydrophobic,” as used herein, refers to a ligand which, as a separate entity, exhibits a higher solubility in a non-aqueous solution (e.g., octanol) than in water.

The term “conjugated protein,” as used herein, refers to a protein complex that includes an apoprotein and one or more associated hydrophobic ligands. The one or more hydrophobic ligands may by covalently or non-covalently associated with the apoprotein. Examples of conjugated proteins include, for example, lipoproteins, glycoproteins, phosphoproteins, hemoproteins, flavoproteins, metalloproteins, phytochromes, cytochromes, opsins, and chromoproteins.

The phrase “mild denaturing,” as used herein refers to a process which reversibly disrupts the secondary, tertiary, and/or quaternary structure of the conjugated protein, thereby facilitating separation of the hydrophobic ligand from the apoprotein. Mild denaturing can be distinguished from harsher conditions, which cleave the peptide backbone, primarily produce insoluble protein upon denaturation/renaturation, and/or disrupt protein structure to a degree such that the protein loses its biological function upon refolding.

The terms “isolating,” “purifying,” and “separating,” as used interchangeably herein, refer to increasing the degree of purity of a polypeptide or protein of interest or a target protein from a composition or sample comprising the polypeptide and one or more impurities (e.g., additional proteins or polypeptides).

The term “haptoglobin” as used herein refers to a protein that is synthesized and secreted mainly in the liver. In blood plasma, haptoglobin (Hp) binds to cell-free hemoglobin (Hb) released from erythrocytes with high affinity and thereby inhibits Hb oxidative activity. The Hp-Hb complex is then removed by the reticuloendothelial system (mostly in the spleen and liver). Hp, in its simplest form, consists of two alpha-beta dimer chains, connected by disulfide bridges, but can exist as polymeric alpha-beta dimer species. The chains originate from a common precursor protein, which is proteolytically cleaved during protein synthesis. Hp exists in two allelic forms in the human population, so-called Hp1 and Hp2, the latter one having arisen due to partial duplication of the Hp1 gene. Three genotypes of Hp, therefore, are found in humans: Hp1-1, Hp2-1, and Hp2-2. Hp of different genotypes have been shown to have similar effects in vivo in attenuating Hb-mediated toxicity. Furthermore, a protein with >90% sequence identity to the Hp1 gene, called haptoglobin related protein (Hpr) also has high affinity for Hb. The term “haptoglobin” thus encompasses all Hp phenotypes (Hp1-1, Hp2-2 and Hp2-1).

Methods

The methods described herein generally involve methods of isolating proteins by membrane filtration. In general, membrane filtration techniques may be divided into three basic categories based on filter pore size and filtration pressure. The first of these categories, known as microfiltration, refers to filters having relatively large pore sizes and relatively low operating pressures. The second category, ultrafiltration, refers to filters having intermediate pore sizes and intermediate operating pressures. Finally, the third category, reverse osmosis, refers to filters having extremely small pore sizes and relatively high operating pressures. Predictably, microfiltration techniques are utilized when large solutes, or species, are to be filtered. Ultrafiltration is used when intermediate species are to be processed, and reverse osmosis is utilized when extremely small species are targeted.

Conventionally, ultrafiltration employs membranes rated for retaining solutes between approximately 1 and 1000 kDa in molecular weight, reverse osmosis employs membranes capable of retaining salts and other low molecular weight solutes, and microfiltration, or microporous filtration, employs membranes in the 0.1 to 10 micrometer (micron) pore size range, typically used to retain colloids and microorganisms.

Traditionally, membrane filters have functioned by placing a porous membrane perpendicularly across the path of a fluid mixture from which a selected species is to be filtered. The fluid mixture flows through the membrane and the selected species is retained by the membrane. Such methods are generally referred to as direct-flow filtration (DFF) or dead-end filtration.

A problem generally associated with DFF is the tendency of the filter to accumulate solutes from the fluid mixture that is being filtered. Accumulation of these solutes creates a layer of retained solutes (known as filter cake) on the filtration membrane and has a tendency to block, or clog, the pores of the membrane decreasing the flow of the fluid mixture, or flux, through the filtration membrane.

The decrease in flux attributable to the accumulation of the solute layer on the filtration membrane may be partially overcome by increasing the pressure differential, or transmembrane pressure that exists across the filtration membrane. Pressure increases of this type are, however, limited in their effectiveness by the tendency of the filter to become increasingly clogged as the filtration process continues. Eventually, of course, further pressure increases become impractical and the filtration process must be halted and the clogged membrane replaced. This is especially true when fragile filtration membranes are employed as they can burst at high operating pressures.

A second problem associated with the accumulation of solutes on the filtration membrane is the tendency for the solute layer to act as a secondary filter. As a result, as the layer of solutes deposited on the filtration membrane increases, passage through the filtration membrane becomes limited to smaller and smaller solutes. The tendency for the solute layer to act as a secondary filter is especially problematic because, unlike the decreased flux attributable to the same layer, it cannot be overcome by increasing the transmembrane pressure.

One solution to the problem of membrane blockage has been the development of tangential-flow filters. Filters of this type employ a membrane which is generally similar to the membrane types employed by traditional filters. In tangential-flow filters, however, the membrane is placed tangentially to the flow of the fluid mixture to cause the fluid mixture to flow tangentially over a first side of the membrane. At the same time, a fluid media is placed in contact with a second surface of the membrane. The fluid mixture and the fluid media are maintained under pressures which differ from each other. The resulting pressure differential, or transmembrane pressure, causes fluid within the fluid mixture, and species within the fluid mixture, to traverse the membrane, leaving the fluid mixture and joining the fluid media.

In operation, the tangential-flow of the fluid mixture over the membrane functions to prevent solutes within the fluid mixture from settling on the membrane surface. This occurs due to the shear force acting on the surface of the membrane due to fluid flow. As a result, the use of Cross-Flow or Tangential-Flow Filtration (TFF) has proven to be an effective means of reducing membrane blockage for membrane filters. Not surprisingly, then, a wide variety of differing designs exist for filters of the tangential-flow type, and TFF methods have been widely described. For example, Marinaccio et al., U.S. Pat. No. 4,888,115 discloses the process (termed “cross-flow”) for use in the separation of biological liquids such as blood components for plasmapheresis. In this process, blood is passed tangentially to (i.e., across) an organic polymeric microporous filter membrane, and particulate matter is removed. In another example of current art, tangential flow filtration has been disclosed for the filtration of beer solutions (Shackleton, EP 0,208,450, published Jan. 14, 1987) specifically for the removal of particulates such as yeast cells and other suspended solids. Kothe et al., (U.S. Pat. No. 4,644,056, issued Feb. 17, 1987) disclose the use of this process in the purification of immunoglobulins from milk or colostrum, and Castino (U.S. Pat. No. 4,420,398, issued Dec. 13, 1983) describes its use in the separation of antiviral substances such as interferons from broths containing these substances as well as viral particles and the remains of cell cultures from which they are derived.

Tangential flow filtration units have also been employed in the separation of bacterial enzymes from cell debris (Quirk et al., 1984, Enzyme Microb. Technol., 6(5):201). Using this technique, Quirk et al. were able to isolate enzyme in higher yields and in less time than using the conventional technique of centrifugation. The use of tangential flow filtration for several applications in the pharmaceutical field has been reviewed by Genovesi (1983, J. Parenter. Aci. Technol., 37(3):81), including the filtration of sterile water for injection, clarification of a solvent system, and filtration of enzymes from broths and bacterial cultures.

The methods described herein can employ direct-flow filtration (DFF), cross-flow or tangential-flow filtration (TFF), or a combination thereof. In certain embodiments, the methods described herein can employ TFF. In particular, the methods described herein can incorporate mathematical modeling steps which can be used to optimize process conditions (e.g., number of diafiltration volumes, the selection of appropriate filtration membranes, or any combination thereof) to afford, for example, a desired degree of target species purity.

For example, provided herein are methods for separating a target species from a solution containing one or more additional species (e.g., one or more impurities). These methods can comprise (i) estimating a retention factor (i.e., the fraction of an individual species retained on the filter membrane) of a target species from the molecular weight of the target species and the retention curve of the filter membrane; (ii) calculating a number of diafiltration volumes needed to afford a desired fraction of target species based on the estimated retention factor for the target species and a net individual species molar flowrate of the target species into the system; and (iii) filtering the solution by ultrafiltration against the filtration membrane having the retention curve from (i) and using the number of diafiltration volumes from (ii), thereby forming a fraction substantially comprising the additional species and another fraction substantially comprising the target species.

By substantially comprising, it is meant that the fraction contains a majority of the component in question following ultrafiltration (e.g., that the fraction contains at least 50% by weight of the component, based on the total weight of the component in the solution pre-ultrafiltration). In some embodiments above, at least 55% by weight (e.g., at least 60%, at least 65%, at least 70%, at least 75%, at least 80%, at least 85%, at least 90%, at least 95%, at least 98%, or at least 99%) of the target species in the solution is present in the fraction substantially comprising the target species.

In some examples, the target species can comprise a target protein, target protein binding molecule, target protein complex, or an impurity (as discussed in more detail below).

The methods can be performed such that the target species is preferentially directed into either the retained (retentate) fraction or the permeate fraction. In some embodiments, the fraction substantially comprising the target species comprises the retained fraction. In other embodiments, the fraction substantially comprising the target species comprises the permeate fraction.

In some embodiments, the fraction of the target species permeated is greater than or equal to 0.90 (e.g., at least 0.90, at least 0.95, at least 0.98, or at least 0.99). In some embodiments, the fraction of the target species permeated is less than or equal to 0.10 (e.g., 0.10 or less, 0.05 or less, 0.02 or less, or 0.01 or less).

In some embodiments, the fraction of the target species retained is greater than or equal to 0.90 (e.g., at least 0.90, at least 0.95, at least 0.98, or at least 0.99). In some embodiments, the fraction of the target species retained is less than or equal to 0.10 (e.g., 0.10 or less, 0.05 or less, 0.02 or less, or 0.01 or less).

In some embodiments, estimation of the retention factor for the target species can be determined from a representative retention curve generated by interpolation or extrapolation of experimentally determined retention factor specifications and/or values from various sized molecules separated on a specified filter membrane. In these embodiments, the experimentally determined retention factor values can be determined experimentally by a user who performs filtering step (iii), determined experimentally by a manufacturer of the filter membrane, provided by a manufacturer of the filter membrane, or any combination thereof. In certain embodiments, the experimentally determined retention factor values can be specifications and/or values provided by the manufacturer of the filter membrane as part of the product specifications for the filter membrane. In some cases, the representative retention curve can exhibit a sigmoidal shape relating a molecule retention factor to a logarithm of molecule size (e.g., molecular weight).

In some embodiments, estimation of the retention factor for the target species can be determined from a representative retention curve generated by fitting a curve to experimentally determined or specified retention factor specifications and/or values from various sized molecules separated on a specified filter membrane. In some cases, the representative retention curve can exhibit a sigmoidal shape relating a molecule retention factor to a logarithm of molecule size (e.g., molecular weight).

In some embodiments, estimation of the retention factor for the target species can be determined using a log normal distribution.

In some embodiments, estimation of the retention factor for the target species can be determined using the equation below:

$$R = \frac{1}{{( \frac{b}{{MW}_{i}} )}^{n} - 1}$$

wherein b and n are regressed from experimental data for the filter membrane, Rᵢ is the retention factor for the target species i, and MWᵢ is the molecular weight of the target species.

In some embodiments, the molecular weight of the target species can be normalized by a representative filter cut-off size that is offset by an experimentally determined value applicable to more than one analogous filter membrane (e.g., two analogous filter membranes, three analogous filter membranes, four analogous filter membranes, five analogous filter membranes, or more). The more than one analogous filter membranes are filter membranes that are formed from the same materials and/or made through the same manufacturing processes, but exhibit different average pore sizes.

In some embodiments, the estimation of the retention factor for the target species can be determined using the equation below:

$${R}_{i , j} = \frac{1}{{( \frac{{MWCO}_{j} + b}{M {W}_{i}} )}^{n} + 1}$$

wherein b and n are regressed from experimental data for a given set of analogous filter membranes. R_{i,j} is the retention factor the target species i. MWᵢ is the molecular weight of the target species, and MWCOⱼ is the molecular weight cut-off (MWCO) of the filter membrane.

The calculating step (ii) can be determined using the equation below:

$$\frac{{C}_{i , V}}{{C}_{i , {V}_{0}}} = \frac{{C}_{i , F}}{{C}_{i , {V}_{0}} ( 1 - {R}_{i} )} + ( 1 - \frac{{C}_{i , F}}{{C}_{i , {V}_{0}} ( 1 - {R}_{i} )} ) {e}^{- ( 1 - {R}_{i} ) {t}_{D}}$$

wherein Rᵢ is the retention factor of the target species estimated from step (i), C_{i,V} is the concentration of species i in a system volume, C_{i,V}_{₀} is the initial concentration of the target species i in the system volume. C_{i,F} is the concentration of the target species i in a feed stream, and t_{D} is the number of diafiltration volumes.

In some embodiments, the target species can comprise a target protein complex, and the calculating step (ii) can be determined using one or more of the equations below:

$$\frac{{dC}_{a / b , V}^{′}}{{dt}_{D}} = [ {C}_{a / b , IN}^{′} - {C}_{a / b , V}^{′} ( 1 - {R}_{a / b} ) ] + ( - \frac{{k}_{b}}{{K}_{D}^{′}} {C}_{a}^{′} {C}_{b}^{′} + {k}_{b} {C}_{c}^{′} ) τ \\ \frac{{dC}_{c , V}^{′}}{{dt}_{D}} = [ {C}_{c , IN}^{′} - {C}_{c , V}^{′} ( 1 - {R}_{c} ) ] - ( - \frac{{k}_{b}}{{K}_{D}^{′}} {C}_{a}^{′} {C}_{b}^{′} + {k}_{b} {C}_{c}^{′} ) τ$$

wherein a and b a target protein and a target protein binding molecule respectively and c represents the target protein complex, C_{i,V}′ is a normalized (e.g., to a reference concentration) concentration of a species (a, b, or c) in a system volume, C_{i,IN}′ is a normalized (e.g., to a reference concentration) concentration of a species (a, b, or c) in a feed stream. Rᵢ is the retention factor of a species (a, b, or c), r is the time for a diafiltration volume, t_{D} is the number of diafiltration volumes, k_{b} is a dissociation rate constant for the target protein complex, and K_{D}′ is a non-dimensionalized dissociation constant for a reaction between species a, b, and c.

In some embodiments, the methods described herein can comprise a batch process. In other embodiments, the method described herein can comprise a continuous process. In these embodiments, the calculating steps can account for changes in species concentrations over time.

In some embodiments, the one or more additional species can comprise an impurity. In these embodiments, the modeling methods can take into account both the target species and the impurity. In this way, modeling can provide for diafiltrations conditions that afford for efficient separation of the target species and the impurity (e.g., conditions which provide for effective separation of the target species from the impurity, such as a desired level of purity of the target species).

In some embodiments, the method can comprise: (i) estimating the retention factor of the target species from the molecular weight of the species and the retention curve of the filter (e.g., using any of the methods of estimation described above); (ii) estimating the retention factor for the impurity from the molecular weight of the impurity and the retention curve of the filter (e.g., using any of the methods of estimation described above); (iii) calculating a number of diafiltration volumes needed to afford a desired fraction of target species and a desired fraction of impurity based on the retention factor of the target species, the retention factor of the impurity, and a net individual species molar flowrate for the target species and impurity (e.g., using any of the methods of estimation described above); and (iv) filtering the solution by ultrafiltration against the filtration membrane having the retention curve from steps (i) and (ii) using the number of diafiltration volumes from step (iii), thereby forming a fraction substantially comprising the impurity and another fraction substantially comprising the target species.

The desired fraction of target species and a desired fraction of impurity can be selected to achieve a desired degree of separation between the target species and the impurity. As with the methods described above, the methods can be performed such that the target species is preferentially directed into either the retained (retentate) fraction or the permeate fraction (with the impurity being preferentially directed into the other fraction. For example, in some embodiments, the fraction substantially comprising the target species comprises a retained fraction and the fraction substantially comprising the impurity comprises a permeate fraction. In other embodiments, the fraction substantially comprising the target species comprises a permeate fraction and the fraction substantially comprising the impurity comprises a retained fraction.

In some embodiments, the fraction of the target species permeated is greater than or equal to 0.90 (e.g., at least 0.90, at least 0.95, at least 0.98, or at least 0.99). In some embodiments, the fraction of the target species permeated is less than or equal to 0.10 (e.g., 0.10 or less, 0.05 or less, 0.02 or less, or 0.01 or less).

In some embodiments, the fraction of the target species retained is greater than or equal to 0.90 (e.g., at least 0.90, at least 0.95, at least 0.98, or at least 0.99). In some embodiments, the fraction of the target species retained is less than or equal to 0.10 (e.g., 0.10 or less, 0.05 or less, 0.02 or less, or 0.01 or less).

In some embodiments, the fraction of the impurity permeated is greater than or equal to 0.90 (e.g., at least 0.90, at least 0.95, at least 0.98, or at least 0.99). In some embodiments, the fraction of the impurity permeated is less than or equal to 0.10 (e.g., 0.10 or less, 0.05 or less, 0.02 or less, or 0.01 or less).

In some embodiments, the fraction of the impurity retained is greater than or equal to 0.90 (e.g., at least 0.90, at least 0.95, at least 0.98, or at least 0.99). In some embodiments, the fraction of the impurity retained is less than or equal to 0.10 (e.g., 0.10 or less, 0.05 or less, 0.02 or less, or 0.01 or less).

In some embodiments, the fraction of the target species retained is greater than or equal to 0.90 (e.g., at least 0.90, at least 0.95, at least 0.98, or at least 0.99) while the fraction of the impurity retained is less than or equal to 0.10 (e.g., 0.10 or less, 0.05 or less, 0.02 or less, or 0.01 or less).

In some embodiments, the fraction of the impurity permeated is greater than or equal to 0.90 (e.g., at least 0.90, at least 0.95, at least 0.98, or at least 0.99) while the fraction of the target species permeated is less than or equal to 0.10 (e.g., 0.10 or less, 0.05 or less, 0.02 or less, or 0.01 or less).

In some embodiments, methods can further comprise increasing the number of diafiltration volumes to increase the fraction of the target species permeated or retained, decrease the fraction of the impurity permeated or retained, or a combination thereof.

In some embodiments, methods can further comprise selecting a filter membrane having a molecular weight cut-off effective to increase the fraction of the target species permeated or retained, decrease the fraction of the impurity permeated or retained, or a combination thereof.

In some embodiments, the method can comprise a method for isolating a target protein from a solution comprising a plurality of proteins that exploit molecular size changes induced by protein complex formation. In these embodiments, modeling can be used to optimize process conditions of one or more of the ultrafiltration separations to aid in the efficient purification of a target species from one or more impurities in a solution (e.g., a crude solution, such as a biological sample).

For example, provided herein are methods that comprise (i) estimating the retention factor of the target protein from the molecular weight of the target protein and the retention curve of a first filtration membrane having a first molecular weight cut-off value; (ii) estimating the retention factor of a first impurity from the molecular weight of the first impurity and the retention curve of the first filtration membrane having the first molecular weight cut-off value; (iii) calculating a first number of diafiltration volumes needed to afford a desired fraction permeated for the target protein based on the retention factor of the target protein from step (i) and a net molar flowrate for the target protein, and a desired fraction retained for the first impurity based on the retention factor of first impurity from step (ii) and a net molar flowrate for the first impurity; (iv) filtering the solution by ultrafiltration against the first filtration membrane using the first number of diafiltration volumes from step (iii), thereby forming a first retentate fraction substantially comprising the first impurity and a first permeate fraction substantially comprising the target protein; (v) contacting the first permeate fraction with a binding molecule that selectively associates with the target protein to form a target protein complex having a molecular weight above the first molecular weight cut-off value of the first membrane; (vi) estimating a retention factor for the target protein complex from the molecular weight of the target protein complex and the retention curve for a second filter membrane having a second molecular weight cut-off value; (vii) estimating a retention factor for a second impurity present in the first permeate fraction from the molecular weight of the second impurity and the retention curve for a second filter membrane having a second molecular weight cut-off value; (viii) calculating a second number of diafiltration volumes needed to afford a desired fraction retained for the target protein complex based on the retention factor of the target protein complex from step (vi) and a net molar flowrate for the target protein, and a desired fraction permeated for the second impurity based on the retention factor of second impurity from step (vii) and a net molar flowrate for the second impurity: and (ix) filtering the first permeate fraction (after contacting it with the targeting protein binding molecule) by ultrafiltration against the second filtration membrane using the second number of diafiltration volumes from step (viii), thereby forming a second retentate fraction substantially comprising the target protein complex and a second permeate fraction substantially comprising the second impurity.

The target protein can be any target protein described below. The binding molecule can be any suitable molecule that selectively associates with the target protein, thereby forming a target protein complex having a molecular weight greater than the target protein (e.g., at least kDa greater than the target protein, at least 25 kDa greater than the target protein, at least 50 kDa greater than the target protein, at least 100 kDa greater than the target protein, or greater).

The term “selectively associates”, as used herein when referring to a binding molecule, refers to a binding reaction which is determinative for the target protein in a heterogeneous population of other similar compounds. Generally, the interaction is dependent upon the presence of a particular structure (e.g., an antigenic determinant or epitope) on the target protein. By way of example, an antibody or antibody fragment selectively associates to its particular target (e.g., an antibody specifically binds to an antigen) but it does not bind in a significant amount to other proteins present in the sample or to other proteins to which the antibody may come in contact in an organism.

In some embodiments, a binding molecule that “specifically binds” a target protein has an affinity constant (Kₐ) greater than about 10⁵ M⁻¹ (e.g., greater than about 10⁶ M⁻¹, greater than about 10⁷ M⁻¹, greater than about 10⁸ M⁻¹, greater than about 10⁹ M⁻¹, greater than about 10¹⁰ M⁻¹, greater than about 10¹¹ M⁻¹, greater than about 10¹² M⁻¹, or more) with that target protein. These values represent desired affinities for binding that may be altered by use of a proper dissociating agent so that the target complex is dissociated when desired. Moreover, other values of affinity constants may be used if binding occurs between most of the target protein and/or target protein complex (e.g., 70% of target protein bound or more, 70% of target protein binding-molecule bound or more)

Examples of suitable classes of binding molecules include, for example, antibodies, antibody fragments, antibody mimetics, proteins (e.g., protein A), peptides, oligonucleotides, DNA, RNA, aptamers, organic molecules, inteins, split-inteins, and combinations thereof. In certain embodiments, the binding molecule comprises an antibody. The term “antibody” refers to natural or synthetic antibodies that selectively bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that selectively bind the target antigen. The term encompasses intact and/or full length immunoglobulins of types IgA, IgG (e.g., IgG1, IgG2, IgG3, IgG4), IgE, IgD, IgM, IgY, antigen-binding fragments and/or single chains of complete immunoglobulins (e.g., single chain antibodies, Fab fragments, F(ab′)2 fragments, Fd fragments, scFv (single-chain variable), and single-domain antibody (sdAb) fragments), and other proteins that include at least one antigen-binding immunoglobulin variable region, e.g., a protein that comprises an immunoglobulin variable region, e.g., a heavy (H) chain variable region (VH) and optionally a light (L) chain variable region (VL). The light chains of an antibody may be of type kappa or lambda.

An antibody may be polyclonal or monoclonal. A polyclonal antibody contains immunoglobulin molecules that differ in sequence of their complementarity determining regions (CDRs) and, therefore, typically recognize different epitopes of an antigen. Often a polyclonal antibody is derived from multiple different B cell lines each producing an antibody with a different specificity. A polyclonal antibody may be composed largely of several subpopulations of antibodies, each of which is derived from an individual B cell line. A monoclonal antibody is composed of individual immunoglobulin molecules that comprise CDRs with the same sequence, and, therefore, recognize the same epitope (i.e., the antibody is monospecific). Often a monoclonal antibody is derived from a single B cell line or hybridoma. An antibody may be a “humanized” antibody in which for example, a variable domain of rodent origin is fused to a constant domain of human origin or in which some or all of the complementarity-determining region amino acids often along with one or more framework amino acids are “grafted” from a rodent, e.g., murine, antibody to a human antibody, thus retaining the specificity of the rodent antibody.

In some embodiments, the method can further comprise calculating the amount of the first impurity present in the first permeate fraction based on the retention factor of the first impurity from step (ii), the net individual species molar flowrate for the impurities into the system, and the first number of diafiltration volumes from step (iii).

In some embodiments, the method can further comprise calculating the amount of the second impurity present in the second retained fraction based on the retention factor of the second impurity from step (vii), the net individual species molar flowrate for the second impurity, and the second number of diafiltration volumes from step (viii).

In some embodiments, the first molecular weight cut-off value can be equal to the second molecular weight cut-off value.

In some embodiments, the first impurity, the second impurity, or any combination thereof can comprise impurities present in the crude sample solution containing the plurality of impurities.

In some embodiments, the second impurity can comprise unbound target protein, unbound binding molecule, or a combination thereof.

In some cases, the target protein complex can be isolated (e.g., if the target protein complex itself is useful, or if the target protein complex is more stable under storage than the target protein).

In other cases, the method can further involve dissociating the target protein complex to re-form the target protein, and isolating the target protein. For example, in some embodiments, the method can further comprise (x) contacting the second retentate fraction with a dissociation agent, thereby inducing dissociation of the target protein complex to yield the target protein and the target protein binding molecule; (xi) estimating a retention factor for the target protein in the second retentate fraction from the molecular weight of the target protein and a retention curve for a third filter membrane having a third molecular weight cut-off value; (xii) calculating a third number of diafiltration volumes needed to afford a desired fraction retained for the target protein based on the retention factor of the target protein from step (xi) and a net molar flowrate for the target protein; and (xiii) filtering the second retentate fraction by ultrafiltration against the third filtration membrane using the third number of diafiltration volumes from step (xii), thereby forming a third retentate fraction and a third permeate fraction substantially comprising the target protein. In some embodiments, the method can further comprise (x) contacting the second retentate fraction with a dissociation agent, thereby inducing dissociation of the target protein complex to yield the target protein and the target protein binding molecule; (xi) estimating a retention factor for the target protein in the second retentate fraction from the molecular weight of the target protein and a retention curve for a third filter membrane having a third molecular weight cut-off value; (xii) estimating a retention factor for a third impurity present in the second retentate fraction from the molecular weight of the third impurity and the retention curve for a third filter membrane having a third molecular weight cut-off value: (xiii) calculating a third number of diafiltration volumes needed to afford a desired fraction retained for the target protein based on the retention factor of the target protein from step (xi) and a net molar flowrate for the target protein, and a desired fraction permeated for the third impurity based on the retention factor of third impurity from step (xii) and a net molar flowrate for the third impurity; and (xiv) filtering the second retentate fraction by ultrafiltration against the third filtration membrane using the third number of diafiltration volumes from step (xiii), thereby forming a third retentate fraction substantially comprising the third impurity and a third permeate fraction substantially comprising the target protein.

The dissociating agent can comprise any suitable agent or agent that stimulates dissociation of the target protein and binding molecule. Suitable dissociating agents are known in the art, and include, for example, pH modifiers (e.g., acids and/or bases), salts, polyelectrolytes, chaotropic agents (e.g., urea, guanidinium chloride), non-aqueous solvents (e.g., alcohols such as ethanol, methanol, isopropanol, butanol, 2-propanol, phenol, or combinations thereof) or combinations thereof.

In some examples, contacting the second retentate fraction with a dissociating agent can comprise contacting the second retentate fraction with an effective amount of a pH modifier to produce an acidic or basic pH, selected so as to facilitate dissociation of the target protein and the binding molecule.

In some cases, contacting the second retentate fraction with a dissociating agent can comprise contacting the second retentate fraction with an effective amount of a pH modifier to produce a pH of 6 or less (e.g., 5.5 or less, 5 or less, 4.5 or less, 4 or less, 3.5 or less, 3 or less, or 2.5 or less). In some embodiments, contacting the second retentate fraction with a dissociating agent can comprise contacting the second retentate fraction with an effective amount of a pH modifier to produce a pH of 2 or more (e.g., 2.5 or more, 3 or more, 3.5 or more, 4 or more, 4.5 or more, 5 or more, or 5.5 or more).

Contacting the second retentate fraction with a dissociating agent can comprise contacting the second retentate fraction with an effective amount of a pH modifier to produce a pH ranging from any of the minimum values described above to any of the maximum values described above. For example, in some embodiments, contacting the second retentate fraction with a dissociating agent can comprise contacting the second retentate fraction with an effective amount of a pH modifier to produce a pH of from 2 to 6, such as from 3 to 6.

In other cases, contacting the second retentate fraction with a dissociating agent can comprise contacting the second retentate fraction with an effective amount of a pH modifier to produce a pH of 8 or more (e.g., 8.5 or more, 9 or more, 9.5 or more, 10 or more, or 10.5 or more). In some embodiments, contacting the second retentate fraction with a dissociating agent can comprise contacting the second retentate fraction with an effective amount of a pH modifier to produce a pH of 11 or less (e.g., 10.5 or less, 10 or less, 9.5 or less, 9 or less, or 8.5 or less.

Contacting the second retentate fraction with a dissociating agent can comprise contacting the second retentate fraction with an effective amount of a pH modifier to produce a pH ranging from any of the minimum values described above to any of the maximum values described above. For example, in some embodiments, contacting the second retentate fraction with a dissociating agent can comprise contacting the second retentate fraction with an effective amount of a pH modifier to produce a pH of from 8 to 11, such as from 8 to 10.

In some examples, contacting the second retentate fraction with a dissociating agent can comprise contacting the second retentate fraction with a non-aqueous solvent, such as an alcohol. Examples of such non-aqueous solvents include, for example, ethanol, methanol, isopropanol, butanol, 2-propanol, phenol, or combinations thereof.

In some examples, contacting the second retentate fraction with a dissociating agent can comprise contacting the second retentate fraction with a chaotropic agent, such as guanidinium chloride, lithium perchlorate, lithium acetate, magnesium chloride, sodium dodecyl sulfate, thiourea, urea, or a combination thereof.

In some examples, contacting the second retentate fraction with a dissociating agent can comprise contacting the second retentate fraction with a dissociation agent (e.g., urea, guanidinium chloride, or a combination thereof) at a concentration of 1 M or more (e.g., 2 M or more, 3 M or more, 4 M or more, 5 M or more) In other embodiments, step (iv) can comprise heating the second retentate fraction to stimulate dissociation of the target protein and binding molecule (e.g., to a temperature of from 40° C. to 60° C.).

Target Species

The target species can comprise any species of interest that can be separated (e.g., from a complex mixture comprising the target species and one or more additional species) by ultrafiltration methods. In some embodiments, the target species can be of biological origin (e.g., the target species can be present in a biological sample, such as a blood or plasma sample or derived from cell cultures). Target species may include, for example, nucleic acids, proteins, lipids, small molecules, carbohydrates, and polymers. In certain embodiments, the target species can comprise a macromolecule (e.g., a biomacromolecule). In certain embodiments, the target species can comprise a conjugated protein.

Examples of target species include, but are not limited to, antibodies (forming an antibody/epitope complex), antigens, nucleic acids (e.g. natural or synthetic DNA. RNA, gDNA, cDNA, mRNA, tRNA, etc.), lectins, sugars (e.g. forming a lectin/sugar complex), glycoproteins, receptors and their cognate target species (e.g. growth factors and their associated receptors, cytokines and their associated receptors, signaling receptors, etc.), small molecules such as drug candidates (either from natural products or synthetic analogues developed and stored in combinatorial libraries), metabolites, drugs of abuse and their metabolic by-products, co-factors such as vitamins and other naturally occurring and synthetic compounds, oxygen and other gases found in physiologic fluids, natural or synthetic toxins, pathogens (e.g., Bacillus anthracis. Yersinia pestis, Francisella tularensis, Coxiella burnetii) other natural products found in plant and animal sources, other partially or completely synthetic products, pathogens (e.g. virus and bacteria, etc.), and the like.

In some embodiments, the target species can have a molecular weight of from 1 kDa to 1000 kDa, such as a molecular weight of from 1 to 250 kDa, a molecular weight of from 1 to 200 kDa, a molecular weight of from 10 to 100 kDa, or a molecular weight of from 50 kDa to 350 kDa.

In some examples, the target species can comprise a target protein, target protein binding molecule, target protein complex, or an impurity. In some examples, the target species can comprise haptoglobin or human serum albumin.

Target species may be found in a variety of heterogeneous test samples (e.g., water, saliva, sweat, urine, serum, blood, plasma, tissues and food). In certain embodiments, the solution from which the target species is separated can comprise a biological sample (e.g., saliva, sweat, urine, serum, blood, plasma, tissues, cell and tissue cultures, or extracts of all of the above).

Ultrafiltration and Filtration Membranes

In the methods described above, the filtration membrane can have a range of pore sizes effective to effect separation of the target species from one or more additional species in a sample (e.g., a pore size which allows the target species to pass through the filtration membrane but retains the additional species, or a pore size which allows the additional species to pass through the filtration membrane but retains the target species). For example, the filtration membrane can be rated for retaining solutes having a molecular weight ranging from the molecular weight of the additional species to the molecular weight of the target species, or from the molecular weight of the target species to the molecular weight of the additional species.

In connection with the methods described herein, ultrafiltration can comprise direct-flow filtration (DFF), cross-flow or tangential-flow filtration (TFF), or a combination thereof. In certain embodiments, the ultrafiltration can comprise tangential-flow filtration (TFF). The membranes useful in the filtration steps described herein can be in the form of flat sheets, rolled-up sheets, cylinders, concentric cylinders, ducts of various cross-section and other configurations, assembled singly or in groups, and connected in series or in parallel within the filtration unit. The apparatus can be constructed so that the filtering and filtrate chambers run the length of the membrane.

Suitable membranes include those that separate the desired species from undesirable species in the mixture without substantial clogging problems and at a rate sufficient for continuous operation of the system. Examples are described, for example, in Gabler FR. Tangential flow filtration for processing cells, proteins, and other biological components. ASM News 1984: 50:299-304. They can be synthetic membranes of either the microporous type or the ultrafiltration type. A microporous membrane has pore sizes typically from 0.1 to 10 micrometers, and can be made so that it retains all particles larger than the rated size. Ultrafiltration membranes have smaller pores and are characterized by the size of the protein that will be retained. They are available in increments from 1000 to 1,000,000 Dalton nominal molecular weight limits.

Generally, the filtration membrane can comprise an ultrafiltration membrane. Ultrafiltration membranes are normally asymmetrical with a thin film or skin on the upstream surface that is responsible for their separating power. They are commonly made of regenerated cellulose, polysulfone or polyethersulfone. In some cases, the filtration membrane can be rated for retaining solutes having a molecular weight of from about 1 kDa to 4,000 kDa, such as from about 1 kDa to about 1,000 kDa or from about 1 kDa to about 500 kDa.

In some cases, each filtration step can involve filtration through a single filtration membrane. In other cases, because membrane filters are not perfect and may have holes that allow some intended retentate molecules to slip through, more than one membrane (e.g., two membranes, three membranes, four membranes, or more) having the same pore size can be utilized for a given filtration step. In these embodiments, the membranes can be placed so as to be layered parallel to each other (e.g., one on top of the other) such that filtered fluid sequentially flows through each of the more than one membrane.

Membrane filters for tangential-flow filtration are available as units of different configurations depending on the volumes of liquid to be handled, and in a variety of pore sizes. Particularly suitable for use in the methods described herein, on a relatively large scale, are those known, commercially available tangential-flow filtration units.

The filtration unit useful herein is suitably any unit now known or discovered in the future that serves as an appropriate filtration module, particularly for microfiltration and ultrafiltration. The preferred filtration unit is hollow fibers or a flat sheet device. These sandwiched filtration units can be stacked to form a composite cell. One example type of rectangular filtration plate type cell is available from Filtron Technology Corporation, Northborough, Mass., under the trade name Centrasette. Another example filtration unit is the Millipore Pellicon ultrafiltration system available from Millipore, Bedford, Mass.

In some embodiments, ultrafiltration may be done with staging to improve separation between retained and filtered solutes and to increase product recovery.

By way of non-limiting illustration, examples of certain embodiments of the present disclosure are given below.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Selective Purification of Proteins Using Size-Based Separation Methods

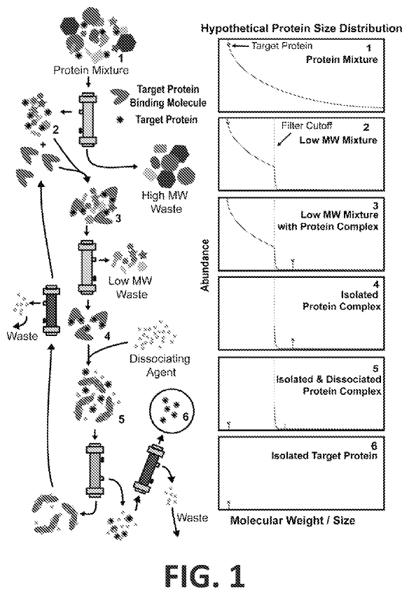

The following example depicts one embodiment of the method illustrated in FIG. 1 as well as the mathematical model used to describe and predict product recovery and purity using the same method.

Similar approaches have been used to isolate enantiomers from racemic protein solutions, but this practice has not been implemented for complex mixtures [1]. Moreover, micellar enhanced ultrafiltration and chelating agents can be used to bind to low MW organic or inorganic species, however this approach lacks specificity and has not been applied to large protein species. Most notably, electrostatic protein-protein interactions have been used in artificial binary protein mixtures to control protein retention (via attractive interactions) and transmission (via the Donnan effect) [2,3]. Although this concept is vital for understanding membrane-based protein separations, it lacks selectivity in isolating a specific protein from a complex protein mixture as impurities may have similar charge to the TP. Moreover, even similarly charged proteins can have attractive electrostatic interactions and the resulting complex may quickly precipitate, further complicating analysis and implementation of this method for complex mixtures.[2] Therefore, we use specific protein-binding interactions to yield a desired soluble TP complex that enables the concept of altering a TP's MW to be used in separating a TP from a complex mixture with high selectivity.

Materials and Methods

Sodium phosphate dibasic, sodium phosphate monobasic, anti-human serum albumin polyclonal antibody in immunoglobulin G (IgG) fraction of rabbit serum, and sodium chloride were purchased from Sigma Aldrich (St. Louis, MO). 0.2 μm Millex-GP PES syringe filters were purchased from Merck Millipore (Billerica, MA). A KrosFlo® Research II tangential flow filtration (TFF) system and hollow fiber (HF) filter modules were obtained from Spectrum Laboratories (Rancho Dominguez, CA). Human fraction IV paste was purchased from Seraplex, Inc (Pasadena, CA). Human serum albumin (HSA) manufactured by OctaPharma (Lachen, Switzerland) was purchased from NOVA Biologics, Inc (Oceanside, CA). Expired units of human red blood cells (RBCs) and thawed human plasma were generously donated by the Transfusion Service in the Wexner Medical Center at The Ohio State University (Columbus, OH).

Hb Purification. Human hemoglobin (Hb) was purified via tangential flow filtration as described by Palmer et al [4]

Human Serum Albumin-IgG Complex Formation. Human serum albumin (HSA) was titrated against various concentrations of anti-HSA immunoglobulin G (IgG) and monitored for HSA-IgG complex formation via size exclusion high performance liquid chromatography (HPLC-SEC). In these mixtures, the HSA concentration was fixed at 0.08 mg/mL, while IgG varied from 0 to ˜0.4 mg/mL.

Purification of HSA-IgG from Artificially Produced Hb and HSA Mixture. HSA (0.08 mg/mL) was mixed with twice the concentration of Hb (0.16 mg/mL). Then IgG was added to the resulting protein mixture at ˜2:1 mass ratio (HSA:IgG) to form the HSA-IgG complex. The resulting mixture was then subject to constant volume diafiltration on a 70 kDa hollow fiber (HF) filter (mPES, 20 cm², C02-E070-05-N) to retain the HSA-IgG complex (13 diafiltration volumes against phosphate buffered saline (PBS) was performed). The yield was determined based on the ratio of the area under the curve of the HPLC-SEC chromatogram at 280 nm (excluding free HSA).

Purification of HSA-IgG from Human Plasma. Human plasma was filtered through a 70 kDa HF filter with 15 diafiltration volumes against PBS. The permeate of the 70 kDa HF filter was concentrated on a 50 kDa HF filter (PS, 20 cm², S02-E050-05-N). Then, approximately 1 mg of IgG was then added to the resulting mixture to form the HSA-IgG complex. The mixture was then re-filtered through a 70 kDa HF filter to isolate the HSA-IgG complex (15 diafiltration volumes against PBS). The yield was determined based on the ratio of the area under the curve of the HPLC-SEC chromatogram at 280 nm (excluding free HSA).

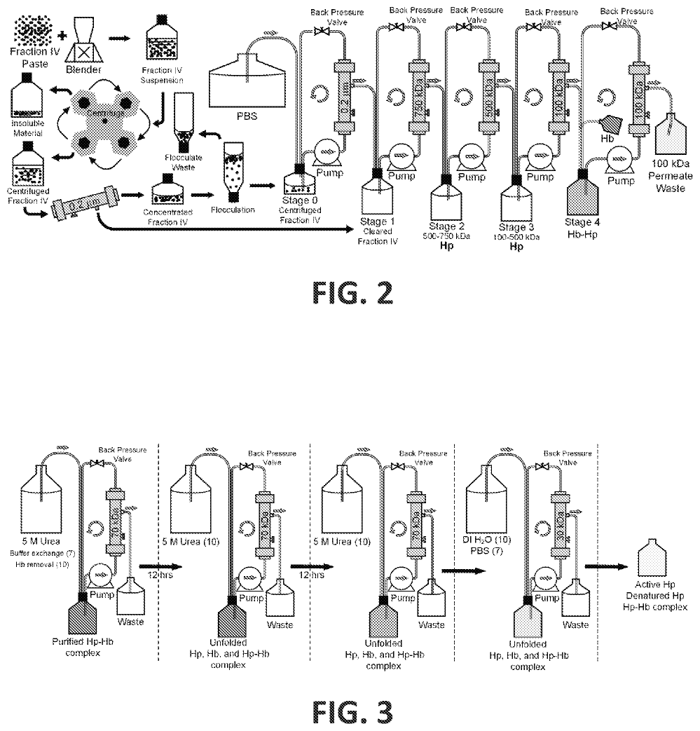

Haptoglobin-Hb Complex Purification from Cohn Fraction IV Based on a recently developed process to purify human haptoglobin (Hp) via TFF, the protein complex purification method was employed to recover the Hb-Hp protein complex from its waste stream [5]. Briefly, 500 g of human Fraction IV (FIV) was suspended then centrifuged to remove insoluble particulates (mostly lipoproteins). The supernatant was concentrated using a 0.2 μm HF filter to 2 L. The retentate was left to rest for 36 hrs to flocculate low density particles, while the filtrate was kept at 4° C. for further processing. After flocculation of the retentate, low density particles in solution were separated. The higher density fraction was then subjected to 10 diafiltration volumes with PBS. The 0.2 μm filtrate was then filtered through a series of HF filters (750, 500 and 100 kDa) as previously described. Hb was then continuously added to the permeate from the 100 kDa HF filter to form the Hb-Hp protein complex, while maintaining the solution with excess Hb to bind all of the Hp in the permeate. The filtrate/Hb mixture was then subjected to 100 diafiltration volumes on a 100 kDa HF filter using fresh PBS to remove excess Hb and low MW proteins. The resulting Hb-Hp complex was then centrifuged for 30 min at 3000 g to remove any insoluble particulates that may had formed during processing. The diagram of the purification process is shown in FIG. 2.

Isolation of Hp from Hb-Hp Complex. To facilitate dissociation of Hb from the purified Hb-Hp complex, 7 mL of Hb-Hp at 2 mg/mL was buffer exchanged (7 diacycles) into a 5 M urea solution at a pH 10 using a 70 kDa HF filter (mPES, 20 cm², C02-E070-05-N). The resulting unfolded protein mixture was then subjected to 10 diacycles using the urea solution with a rest period of 12 hr in between processing to yield a total of 30 diacycles. The solution was then diafiltered for 10 diacycles into deionized (DI) water followed by 7 diacycles into PBS using a 30 kDa HF filter (mPES, 20 cm², C02-E030-05-N). The schematic of this process is shown in FIG. 3.

Analytical Size Exclusion Chromatography. Hp fractions were separated via analytical size exclusion high performance liquid chromatography (HPLC-SEC) using an Acclaim SEC-1000 (4.6×300 mm) column (Thermo Fisher Scientific, Waltham, MA) attached to a Dionex UltiMate 3000 system (Thermo Fisher Scientific, Waltham, MA) as described previously [5].

Hb Concentration. The concentration of Hb was measured spectrophotometrically using a HP 8452A Diode Array Spectrophotometer (Hewlett Packard, CA) via the Winterbourn equations[6].

Gel Electrophoresis. The purity of Hb-Hp and Hp was analyzed via SDS-PAGE using an Invitrogen Mini Gel Tank (Thermo Fisher Scientific, Waltham, MA). Samples was prepared according to the manufacturer's guidelines. Gels were loaded with approximately 30 μg of protein per lane and tested under reducing (via addition of 0.1 M dithiothreitol) and non-reducing conditions. Densitometry was performed using ImageJ.

Hb Binding Capacity of Hp. The Hb binding capacity (HbBC) of Hp samples was determined based on the fluorescence quenching method described in the literature using a PTI Fluorometer (Horiba Scientific, NJ)[7].

Mathematical Model of TFF. A mathematical model was developed to describe the separation of molecules via TFF. Briefly, the system (retentate vessel, TFF filter and tubing) was assumed to be well mixed (i.e. permeate flow rate was much lower than the pump flow rate, leading to negligible concentration difference between inlet and outlet of HF filter) and at constant volume (V). Furthermore, the feed flow rate (F) from the feed reservoir into the retentate vessel was assumed to be constant which, based on a constant system volume, led to the permeate flow rate (P) being equal to the feed flow rate (F). Moreover, the permeate concentration of each species was determined based on Equation 1.

C_{i,P}=C_{i,V}*(1−Rᵢ) (1)

In which C_{i,P} is the concentration of species i in the permeate, C_{i,V} is the concentration of species i in the retentate vessel and Rᵢ is the fraction of species i retained on the membrane (i.e. retention factor). Based on these assumptions and without considering any chemical reactions, the species concentration over time was determined from Equation 2.

$$\frac{{dC}_{i , V}}{dt} = ( {C}_{i , F} - {C}_{i , P} ) * F / V \qquad ( 2 )$$

In which C_{i,F} is the concentration of species i being fed into the retentate vessel and t is the process time. Normalizing the process time by the time for the completion of one diafiltration volume (τ=V/F), the change in concentration per diafiltration volume yields the simplified relationship shown in Equation 3.

$$\frac{{dC}_{i , V}}{{dt}_{D}} = ( {C}_{i , F} - {C}_{i , P} ) = ( {C}_{i , F} - {C}_{i , V} * ( 1 - {R}_{i} ) ) \qquad ( 3 )$$

In which t_{D} is the dimensionless time equal to the number of diafiltration volumes (t_{D}=t/τ). Equation 3 can be analytically solved for a system initially charged with C_{i,V}_{₀} to yield the fraction of species i retained in the system (C_{i,V}/C_{i,V}_{₀}) as shown in Equation 4.

$$\frac{{C}_{i , V}}{{C}_{i , {V}_{0}}} = \frac{{C}_{i , F}}{{C}_{i , {V}_{0}} ( 1 - {R}_{i} )} + ( 1 - \frac{{C}_{i , F}}{{C}_{i , {V}_{0}} ( 1 - {R}_{i} )} ) {e}^{- ( 1 - {R}_{i} ) {t}_{D}} \qquad ( 4 )$$

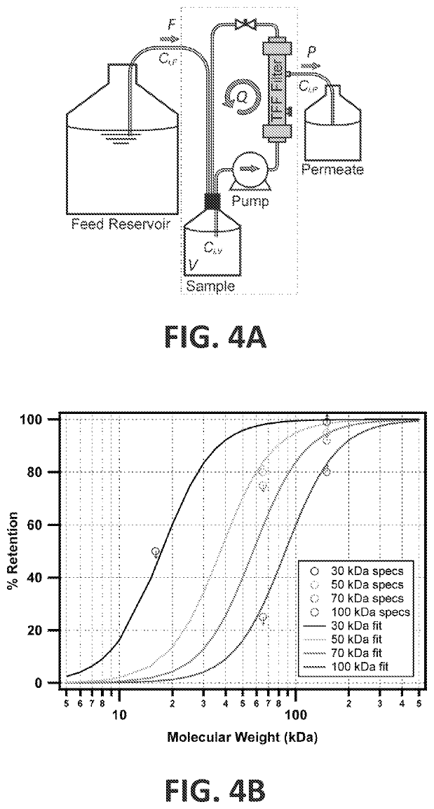

To model this system, it was necessary to obtain the retention factor for each of the species being modeled (Rᵢ). This was determined based on the manufacturer's specified retention values for TFF filters with MWCOs of 30, 50, 70 and 100 kDa. Based on these values, the Hill equation was used to model the S-shaped retention curves as shown in Equation 5.

$${R}_{i , j} = \frac{1}{{( \frac{{MW}_{{50}_{j}}}{{MW}_{i}} )}^{n} + 1} = \frac{1}{{( \frac{{MWCO}_{j} + b}{{MW}_{i}} )}^{n} + 1} \qquad ( 5 )$$

In which MW₅₀_{ⱼ} is the MW that corresponds to 50% retention for a particular filter j, n in the Hill coefficient (i.e. steepness of the curve) and MWᵢ is the MW of the species i. The assumptions used to determine values for MW₅₀ and n were that all HF filters had the same Hill coefficient (n) and that the MW₅₀_{ⱼ} for a filter was determined based on the difference between its specified MWCOⱼ and a parameter b that was equal for all HF filters. A non-linear least squares regression was performed in Excel to determine b and n which yielded values of −12.6 kDa and 2.96, respectively. A diagram of the modeled TFF system, and the manufacturer specifications for each filter with their corresponding fitted retention curves are shown in FIG. 4.

For modeling of HSA-IgG separation, the MW of HSA was set to 65 kDa and the MW of the HSA-IgG complex was set to 220 kDa. For modeling of the Hb-Hp separation, the MW of tetrameric Hp was set to 210 kDa while the MW of the tetrameric Hb-Hp complex was set to 340 kDa.

Results and Discussion

HSA-IgG Complex Purification

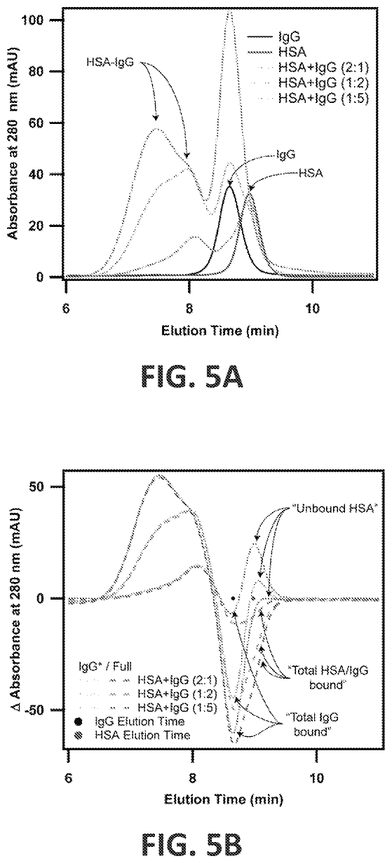

In the initial studies, one aim was to recover human serum albumin (HSA) using an immunoglobulin G (IgG) that was specific to HSA. Thus, based on the terminology presented in FIG. 1, HSA was the TP and IgG was the TPBM. However, to design the experiment, we first sought to characterize the HSA-IgG (TP-TPBM) complexes formed when the two components (TP and TPBM) were mixed. These results would aid in determining the MWCO of the HF filter to use for isolation of the TP-TPBM complex and what concentrations of HSA and IgG would yield the largest amount of bound HSA molecules per IgG molecule with minimal unbound HSA. Since a polyclonal IgG was used, it would be expected that at low IgG to HSA mass ratios, IgG would bind to a single HSA molecule, leading to efficient binding of HSA, but with unbound HSA still remaining in solution. Conversely, at high IgG to HSA mass ratios, all HSA molecules would be bound, but more than one IgG could bind to each HSA molecule (via binding to different epitopes on the HSA surface). Thus, the polyclonal IgG used in this study was mixed at varying mass ratios with HSA to analyze the size of the HSA-IgG complexes formed and a preferred mass ratio for HSA recovery. The resulting mixtures were then separated via HPLC-SEC and the results are shown in FIG. 5.

As shown in FIG. 5, at the concentrations used in this study, a 2:1 HSA to IgG mass ratio resulted in most of the HSA existing in an unbound state. When the mass of IgG was double that of HSA, almost all the HSA was complexed with IgG. Further, at five times the mass of IgG compared to HSA, no free HSA was detected in solution. Moreover, it was possible to note the formation of at least two distinct HSA-IgG complexes (indicated in FIG. 5A). The first eluted at ˜8 minutes (˜300-400 kDa) while the second eluted at ˜7.5 minutes (˜700-800 kDa). It was likely that these MW estimates were overestimates, since the HSA-IgG species do not have a spherically compact structure, leading to a larger overall apparent size shown in the HPLC-SEC chromatogram. Thus, it was concluded that the first complex corresponded to a single IgG molecule bound by one or two HSA molecules (˜200-250 kDa), while the latter corresponded to a complex with more than one IgG molecule. Although no other distinct peaks were observed, the SEC column used in this study was limited by a separation range of up to 1,000 kDa which prevented proper separation of larger order complexes on the chromatogram, which could have also formed.

These observations agreed with the expected behavior as formation of the single IgG complex was favored when high HSA to IgG mass ratios were used, while the higher order complexes were favored when more IgG was added. Therefore, since at a mass ratio of 2:1 (HSA:IgG), most of HSA was already bound, further increasing the mass of IgG likely lead to formation of higher order HSA-IgG complexes, consuming more IgG per bound HSA. More importantly, based on FIG. 5 it was determined that the IgG-HSA complexes formed had MWs larger than at least 200 kDa (IgG-HSA complex). Therefore, it was expected that a HF module with MWCO of 70 kDa would be able to sufficiently retain the HSA-IgG complexes, while permeating HSA and small amounts of unbound IgG.

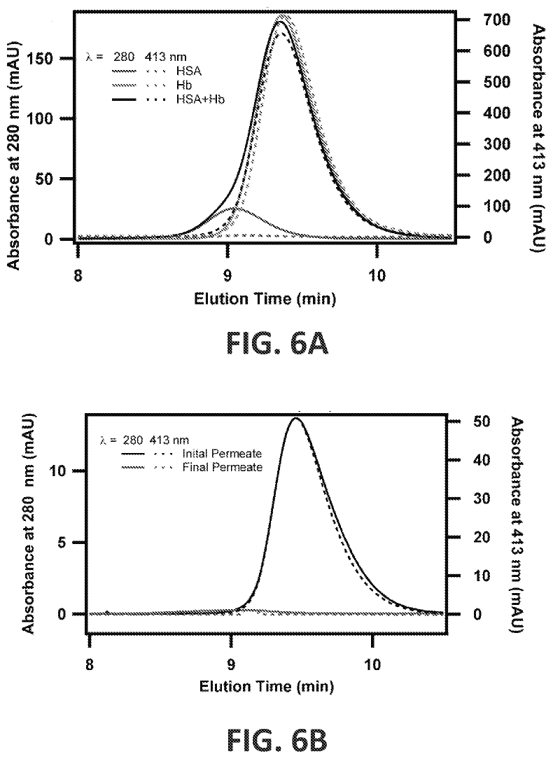

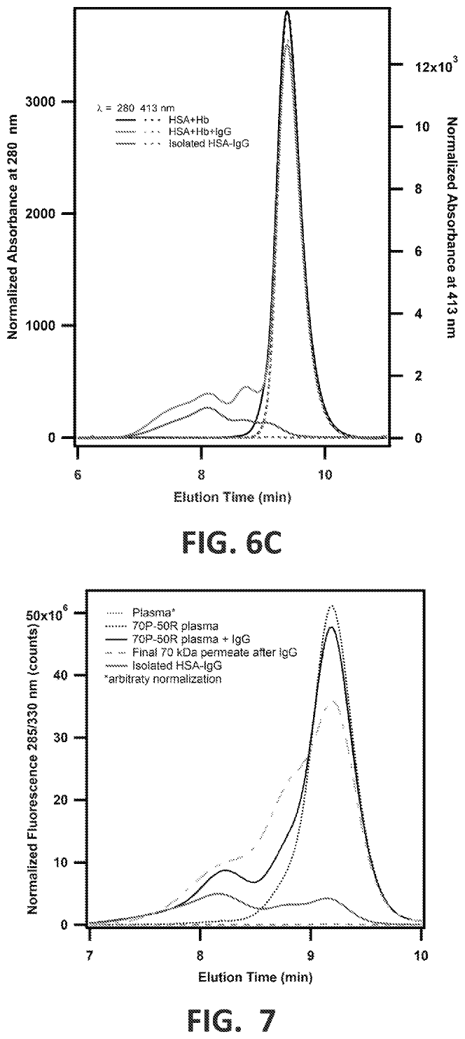

After characterizing the HSA-IgG complexes, our first step in validating the selective TFF strategy in FIG. 1 was to demonstrate the recovery of the TP-TPBM complex via IgG (TPBM) binding to HSA (TP) in an artificial mixture composed of HSA and hemoglobin (Hb). Hb (64 kDa) and HSA (66 kDa) have similar MWs (an indicator of molecular size), which imply that they could not be separated via conventional TFF. However, by using IgG as a TPBM, the HSA-IgG complex may be isolated from Hb, yielding the purified HSA-IgG complex. Hb was chosen as the impurity in HSA purification as it has a characteristic high absorption band (Soret peak) which facilitated tracking of the impurity (i.e. the Hb). In this scenario, the protein mixture was already composed of low MW species, thus pre-filtration of the mixture to retain larger MW impurities was not performed. Based on the results of FIG. 5, a ˜1:2 (HSA:IgG) mass ratio was chosen to form the HSA-IgG complex, since at this ratio, most of HSA bound to IgG while the formation of higher order IgG complexes was minimized. After mixing HSA and Hb and adding anti-HSA IgG, the resulting mixture was subjected to constant volume diafiltration on a 70 kDa HF membrane to isolate the HSA-IgG complex. The results from this analysis are shown in FIG. 6.

As mentioned previously. HSA (66 kDa) and Hb (64 kDa) have similar MWs, but they differed in their elution times due to their shape (Hb is a more compact sphere compared to HSA, leading to a higher elution time). In the HPLC-SEC chromatogram, the mixture yielded an almost uniform peak (FIG. 6A and FIG. 6C) when monitoring the absorbance at 280 nm. However, Hb has a strong absorbance in the Soret region, while HSA does not, easily facilitating the detection of Hb in the mixture (FIG. 6A). After addition of IgG to the mixture, the HSA-IgG complex was formed (FIG. 6C). Moreover, the initial permeate from the diafiltration process was found to contain primarily Hb as the 280 and 413 nm peaks overlapped (FIG. 6B). On the other hand, after 13 diafiltration volumes, the permeate was practically cleared of Hb with minimal amounts of protein permeating through the filter. Thus, as shown in FIG. 6C, the final isolated HSA-IgG complex contained no detectable level of Hb.

In addition to confirming that no detectable level of Hb was present, based on the overall recovered HSA-IgG complex chromatogram, more than 50% of the complex was recovered. Unfortunately, some of the >200 kDa species were lost, which would not be expected to easily permeate through the 70 kDa HF filter. These species were likely lost either through general processing (unspecific binding to the filter or retained in the tubing) or some of the HSA-IgG complexes may have precipitated (either due to TFF processing or via aggregation of large immune complexes). Moreover, the equilibrium between the HSA-IgG complex and unbound proteins could have contributed to the loss of HSA and some IgG since the dissociation constant of rabbit polyclonal anti-HSA IgG has been shown to be on the order of 10⁻⁸ M which is almost of same order of magnitude as the concentration of IgG used in this study (10⁻⁷ M)[8]. Moreover, the heterogeneity of polyclonal antibodies can lead to dissociation constants ranging from 10⁻⁴ M to 10⁻¹² M. Thus, the HSA-IgG complex in equilibrium with unbound HSA and IgG may have continuously shifted towards the unbound components as HSA was filtered out of the system, thus reducing the amount of HSA-IgG complex[9].

Even though the retained HSA-IgG complex on the SEC-HPLC chromatogram showed the presence of low MW species that would be expected to permeate through the 70 kDa HF filter, based on the low protein content of the permeate, further diafiltration was ineffective. In this experiment, the protein load on the membrane was low, thus fouling of the membrane was unlikely. Thus, the presence of these low MW species in the chromatogram may be due to the dissociation of the complexes in equilibrium with the individual proteins in the complexes, while running them on the HPLC-SEC column[10-13]. As mentioned above, this dissociation may have been favored due to the low concentrations of IgG used. Further, even though the antigen-antibody reaction favors complex formation, it has been shown that, depending on the dissociation rate constant, it is possible for the complex to dissociate during analysis in the SEC column[13,14]. Finally, filtration of the same HSA and Hb mixture through the 70 kDa HF filter without addition of IgG lead to permeation of practically all the protein within ˜10 diafiltration volumes. Thus, the HSA-IgG complex could not be further purified.

Overall, in this first experiment, HSA-IgG was successfully isolated from a simple mixture of HSA and Hb (FIG. 6). Next, we sought to demonstrate how to purify a protein in its complexed form from a complex mixture. Since the separation of HSA-IgG had already been demonstrated, the same TP and TPBM pair was used to isolate HSA-IgG from plasma, a complex protein mixture. Unlike the simple HSA and Hb mixture, plasma contained large MW impurities. Thus, plasma was first diafiltered on a 70 kDa membrane to permeate HSA while retaining large MW species (i.e. permeating the TP and low MW impurities). The permeate fraction from the 70 kDa membrane was concentrated on a 50 kDa HF filter to maintain a constant operating volume, forming a fraction between 70 and 50 kDa (bracket between permeate of 70 kDa HF filter and retentate of 50 kDa HF filter: 70P-50R). Then, IgG was added to the 70P-50R fraction to form the HSA-IgG complex, yielding a mixture of HSA-IgG complex and low MW impurities. The HSA-IgG complex was then selectively retained by subsequent diafiltration on the 70 kDa membrane. The results from this experiment are shown in FIG. 7.

As shown in FIG. 7, plasma is a complex protein mixture with a wide range of MWs. Filtration of plasma through the 70 kDa HF filter retained most of the large MW species since plasma contained species at ˜8 min elution time that were not present on the 70P-50R sample. Addition of IgG to 70R-50P led to the formation of HSA-IgG complexes with a similar elution chromatogram to that of the 2:1 (HSA:IgG) mass ratio shown in FIG. 5. This was expected as the amount of IgG added was much lower than the expected mass of HSA in the permeate.

Similar to the example demonstrating isolation of HSA from the Hb and HSA mixture (FIG. 6), ˜50% of the complex was recovered. Moreover, compared to FIG. 6, a similar HSA-IgG chromatogram was observed since there were small MW species expected to permeate through the 70 kDa HF filter that appeared in the final HSA-IgG chromatogram. However, similar to the experiment in FIG. 6, practically no protein permeated through the filter as shown in the final permeate chromatogram in FIG. 7. To ensure that filter fouling was not the cause for retention of these species, the filter was cleaned with 0.1 M sodium hydroxide prior to re-diafiltering the complex. Moreover, even when a new filter was used, there was not significant removal of protein from the retained HSA-IgG complex. Thus, it was likely that the complex dissociated as it passed through the analytical HPLC-SEC column or that the HSA-IgG equilibrium allowed for the presence of pure HSA in solution.

Overall, with the HSA-IgG complex purification scheme, it was demonstrated that the protein complex TFF method could be used to isolate protein complexes. The products had high purities (based on the absence of Hb or lack of protein permeation through the HF filter), but protein complex recovery was limited by the dissociation of the HSA-IgG complex during processing. Moreover, for isolation of pure HSA, HSA could be dissociated from the HSA-IgG complex as previously described in the literature via the use of chaotropic agents or low pH incubations[9,15].

Hb-Hp/Hp Purification from Cohn Fraction IV

After assessing that protein complexes could be purified with the methodology described in this study, we next sought to test this purification strategy using a practical example. In our lab, we purify large amounts of Hb and Hp using TFF [4,5]. Hp is a plasma glycoprotein with the main role of scavenging toxic cell-free Hb in blood. During conditions in which red blood cells lyse (i.e. hemolysis), plasma Hp quickly binds to Hb, preventing the toxic side-effects of cell-free Hb. However, during extensive hemolysis, plasma Hp levels are depleted, allowing cell-free Hb to elicit oxidative tissue damage and systemic hypertension. Therefore, Hp replacement therapy may be used to treat these states of hemolysis by binding to cell-free Hb in plasma to detoxify it. However, in the Hp purification process, approximately 50% of Hp initially present in human Cohn fraction IV (FIV) is lost as it is not retained within the TFF system. Thus, we wanted to recover this Hp via the selective TFF method described in this work. Moreover, with the large volumes of Hp and Hb being processed, we were able to perform dissociation studies to separate Hb and Hp from the Hb-Hp complex, to prove that we can recover the individual proteins (Hb and Hp) from the Hb-Hp protein complex.

As shown in FIG. 2, the last HF filter used for Hp purification has a MWCO of 100 kDa. In this scenario, the Hp purification process retained any large MW impurities from FIV, leaving only Hp and low MW impurities on the 100 kDa permeate. Given the high binding affinity of Hp for Hb, Hb was used as a TPBM to bind to Hp (the TP) that permeated through the terminal 100 kDa filter of the Hp purification process. Then the Hb-Hp containing permeate was re-diafiltered through a 100 kDa HF filter to isolate the Hb-Hp complex. Similar to isolation of HSA-IgG from the HSA and Hb mixture, use of Hb as the TPBM has the advantage of possessing a Soret peak which allows for tracking of the Hb-Hp complex. Moreover, the Hb-Hp complex itself has potential biomedical applications given that it could be used to target CD163+ macrophages and monocytes[16,17].

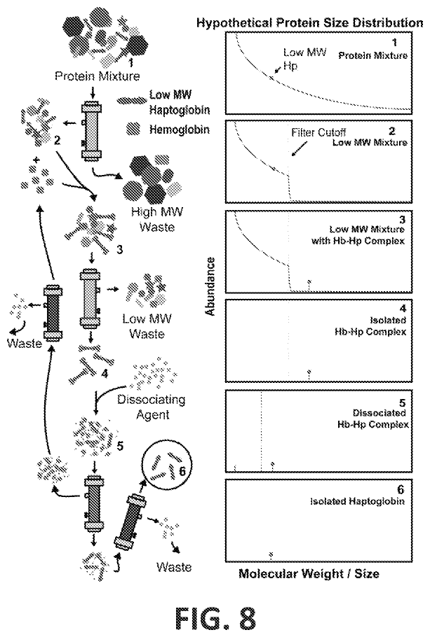

Based on this idea, excess Hb was added to the 100 kDa permeate of the process used to isolate Hp (FIG. 2) described in the Methods Section to form Hb-Hp complexes. Then, the Hb and permeate mixture containing Hb-Hp was refiltered through a 100 kDa filter to isolate the Hb-Hp complex. Dissociation of the complex was later performed using 5 M urea as the dissociating agent, which allowed for partial removal of Hb from Hb-Hp. The overall procedure used in this experiment is illustrated in FIG. 8.

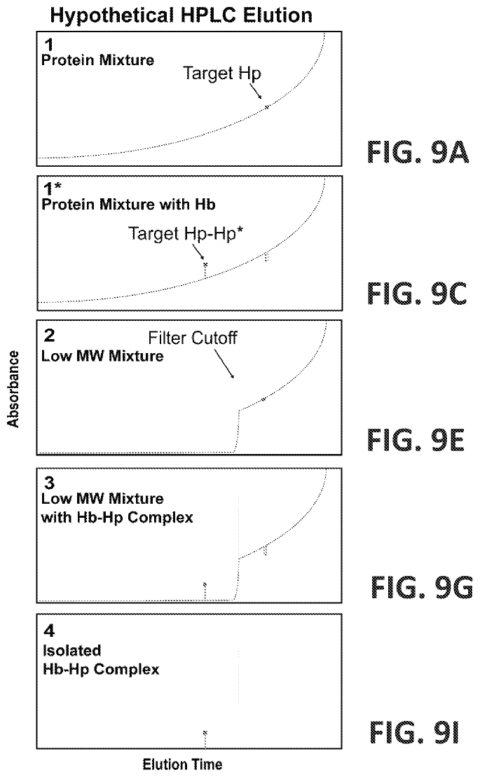

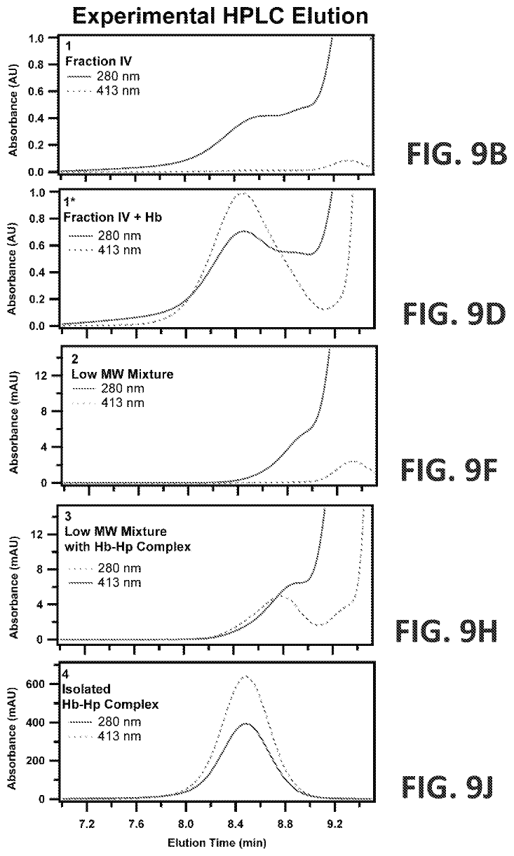

During the purification of the Hp-Hb complex, samples were taken at different stages of the process. These samples were analyzed on an HPLC-SEC column and the results were compared to the theoretically predicted separation based on the schematic in FIG. 1. The comparison of predicted versus experimental results are shown in FIG. 9.

From these results, the addition of Hb to Cohn Fraction IV increased the amount of large MW species (compare 1 to 1*). These species matched our purified product (compare 1* to 4). Furthermore, permeate analysis (2) showed that the unbound Hp did not easily permeate through the 100 kDa HF filter. This can be seen due to a lower relative abundance of the Hb-Hp complex when Hb was added to the protein mixtures (compare the amount of Hb-Hp in 1* to 3). Another observation was that, by comparing 2 to 4, it was noticeable that some of the Hb-Hp complex was capable of permeating through the 100 kDa HF filter. This was not surprising as Hb only added 64-96 kDa to the low MW Hp species (dimers and trimers). Thus, the change in MW from Hb binding was not sufficient for full retention of the complex on the 100 kDa membrane. Yet, even though some of the complex was lost, we were able to isolate some of the Hb-Hp and demonstrate the application of this purification strategy. Moreover, based on the purified complex elution time, the Hb-Hp complex had a MW of ˜350 kDa which indicated it was an average of ˜220 kDa for the pure Hp (approximate MW for tetrameric Hp without any bound Hb) which indicated that most Hp trimers and dimers were lost during filtration.

Using this method, the resulting Hb-Hp complex isolated contained 200 mL of 2.2 mg/mL Hb as determined from its Soret peak absorbance. Based on the expected Hb binding capacity present in the 100 kDa permeate stage of the Hp purification process (˜3 g), the recovery of Hp was approximately 10-15%. The low recovery rate was attributed to the loss during general processing and from loss of product that permeated through the 100 kDa HF filter. The equilibrium between the Hb-Hp protein complex and the individual protein components (Hb and Hp) in the complex was not expected to be a factor during the separation, since the Hb-Hp complex has a dissociation constant in the picomolar range[18,19].

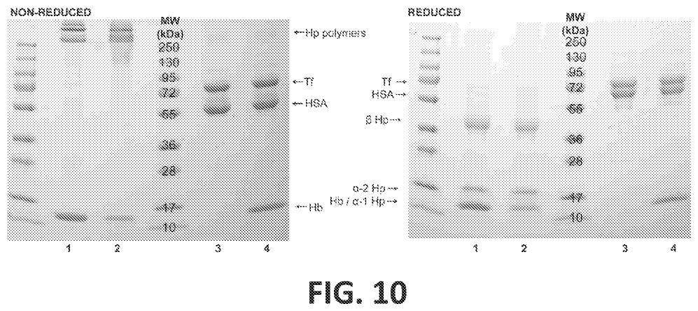

Starting with the purified Hb-Hp complex, Hb was dissociated as described in the Methods Section to isolate Hp (FIG. 3). The SDS-PAGE of the purified Hb-Hp complex and recovered Hp from the complex is shown in FIG. 10.

From the SDS-PAGE analysis, practically no impurities could be detected (>95% pure based on densitometry) in the purified Hp-Hb complex. The purification process effectively removed the low MW impurities and retained only the Hp polymers (compare Lane 4 to 1). Furthermore, from both the SDS-PAGE band intensity analysis and the spectrophotometrically determined amount of Hb bound to the purified Hp species, the Hp to Hb mass binding ratio was estimated to be ˜1.6:1. This is approximately the same theoretical mass binding ratio assuming one Hp2-2 dimer (expected to be the predominant Hp phenotype in Cohn Fraction IV) is bound to one Hb αβ dimer. Moreover, as shown on the SDS-PAGE most of the Hp that permeated through the 100 kDa consisted of ˜250 kDa Hp polymers, which was similar to the HPLC-SEC estimates of ˜220 kDa from FIG. 9.

Urea treatment of the Hb-Hp complex provided experimental proof of principle that the protein complexes could be dissociated to yield the isolated TP. Unfortunately, urea treatment was not successful in fully removing the bound Hb in the Hb-Hp complex as shown by the presence of residual Hb in Lane 2 of the SDS-PAGE. Yet, it was possible to note a decrease in the Hb band intensity in the urea treated Hb-Hp complex. Furthermore, using the 1.6:1 (Hp:Hb) mass binding ratio, SDS-PAGE analysis indicated that about 20% of the Hp was still bound to Hb as the Hp-Hb complex. In comparison, using total protein and spectrophotometric analysis, the product consisted of 25% active Hp (based on fluorescence Hb binding assay), 29% Hb-Hp complex and 52% inactive Hp (denatured). Furthermore, compared to the starting Hb-Hp complex, 52% of Hp was lost during processing, 12% was active, 13% remained bound to Hb and 23% was denatured. Overall, these results could be greatly improved through optimization of the protein unfolding conditions to avoid protein denaturing and using a lower MWCO HF filter for the diafiltrations to avoid loss of protein. However, the process to obtain the purified Hp exemplifies how to use TFF for isolation of a TP from the TP-TPBM complex.

Mathematical Modeling

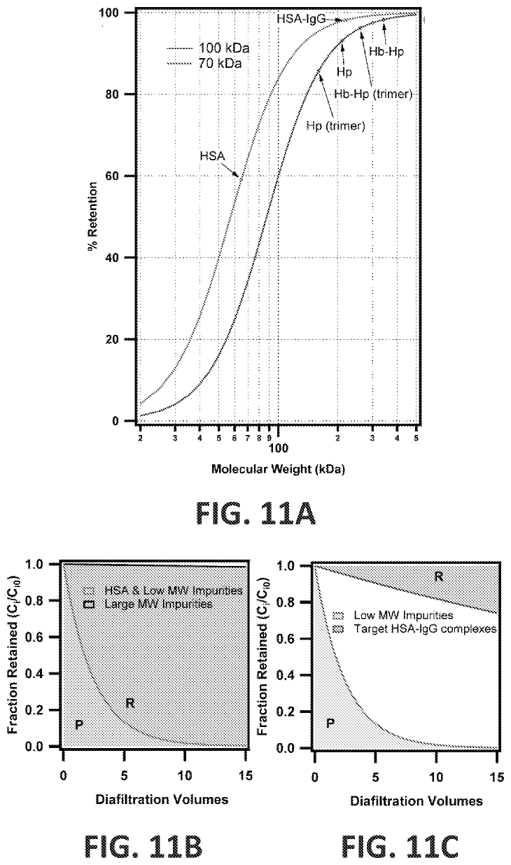

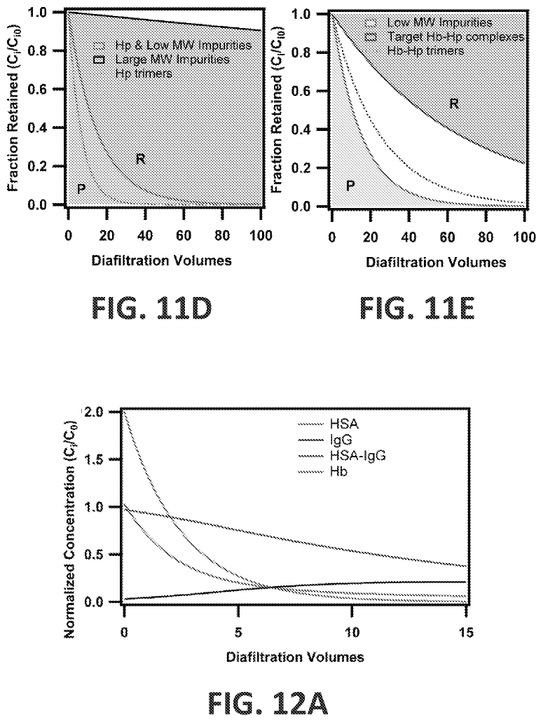

In order to better understand and predict the performance of the TFF separation process, the retention curves for the 70 and 100 kDa HF filters were estimated and the mathematical model discussed in the Methods Section was employed. The results from the mathematical model for the two separation experiments performed earlier are shown in FIG. 11.

TFF filters have a nominal MWCO which rates performance of the filter for purification of different sized macromolecules. This MWCO is determined based on the retention curve of each filter (FIG. 11A). These retention curves relate the MW of the species to their percentage retention on the HF membrane. This curve is only an estimate, since the main determinants for filtration are the size and shape of the molecules, not the species mass. However, for modeling of the filtration system, estimates for the retention of the species to be separated were required. Thus, the manufacturer's specific retention ratings for various membranes (30, 50, 70 and 100 kDa mPES filters) was used to determine the best fit coefficients to the Hill equation. The Hill equation was chosen as it provides a good fit to the characteristic sigmoidal shape of membrane retention curves on a logarithmic axis[20,21]. The fits for some example retention curves and description on how the retention curves were determined can be found in the Methods Section (FIG. 4).

Based on the retention curves, the TFF systems used for isolation of HSA-IgG (FIG. 11B and FIG. 11C) and Hb-Hp (FIG. 11D and FIG. 11E) were modeled. As shown in FIG. 11B, ˜10 diafiltration volumes were required to permeate all (>99%) HSA through a 70 kDa HF filter which was observed experimentally when only HSA and Hb were mixed. Moreover, FIG. 11C demonstrated how the current MWCO filter led to loss of the HSA-IgG complex, which explains some of the protein loss observed experimentally. Yet, from the model, it was expected that >75% of the HSA-IgG would be retained, but only ˜50% was retained experimentally. This difference was likely due to the equilibrium between the HSA-IgG complex and pure HSA and IgG species which this simple model did not account for (the effect of reaction equilibrium is further discussed later in this section).

For the isolation of Hb-Hp, the model demonstrated how trimeric Hp-Hb would not have been retained over the 100 diafiltration volumes performed (FIG. 11E). This confirmed the experimental results which indicated that tetrameric Hp consisted of most of the retained Hp. Furthermore, a substantial loss of protein was observed for the Hb-Hp complex, with only ˜20% retained at the end of processing (based on the MW of tetrameric Hp). This value was in close agreement to the experimental value of 10-15%. Moreover, the model did not account for the processing required to obtain the 100 kDa Hb-Hp permeate. During the Hp purification process. Stage 3 (FIG. 2) was diafiltered for 100 diafiltration volumes which yielded a large volume of permeate. Thus, to maintain a constant volume system in Stage 4, Hb was continuously added and the mixture was diafiltered on a 100 kDa HF filter. The filtration performed during this time did not count towards the 100 diafiltration volumes performed on the Hb-Hp complex shown in FIG. 11. Thus, the additional diafiltration volumes would add ˜10-20 more diafiltration volumes to the Hb-Hp complex purification, explaining why the model predicted a higher retention that what was observed experimentally. The accuracy of the model predictions without inclusion of the equilibrium reactions was likely due to the practically irreversible reaction of Hb to Hp[22], with dissociation constant much lower than that of an immunoglobulin binding to its antigen (<10⁻¹² M [18,19] for Hb-Hp compared to ˜10⁻⁸ M for an antigen-antibody).

The difference in the number of required diafiltration volumes for separation of the complex from the impurities or TP can be visualized on the retention curve. For the HSA-IgG process, HSA was found close to the middle portion of the retention curve while HSA-IgG was at >90% retention. This allowed for effective permeation of HSA or low MW impurities without loss of the protein complex. On the other hand, both Hp and Hb-Hp complex were observed at >90% retention, which made it difficult to permeate the molecules. Interestingly, even in this case, we were able to experimentally purify the Hb-Hp complex. In preferred scenarios, the TP and TPBM would be at the two opposing extremes of the sigmoidal retention curve, allowing for filtration of the TP and low MW impurities while retaining the TP-TPBM complex.

The model presented here also demonstrated how some of the large MW species could contaminate the isolated protein complex. Even though these large MW species would have high retention (assumed to be 99.9% retained in the model), at a sufficient number of diafiltration volumes, they could permeate significantly thorough the filter prior to the addition of the TPBM. However, for systems such as HSA-IgG in which the TP is smaller than the TPBM, dissociation of the TP-TPBM complex and separation of the TP from the TPBM would leave the impurities retained with the TPBM. Moreover, in general cases that do not require many diafiltration volumes, only small amounts of large MW complexes would be expected to permeate the filter. Furthermore, if a sufficiently large TPBM is utilized, then a larger MWCO HF filter relative to the MWCO used to permeate the TP can be used to retain the TP-TPBM complex. Thus, the large MW species (relative to the first MWCO HF filter) mixed with the TP-TPBM complex could permeate through the HF filter, leaving only purified TP-TPBM protein complex in the retentate.

In addition to assessing the specific TP and TPBM pairs discussed in this work, the simple mathematical model can predict the performance of the separation system on different TP and TPBM pairs. For example, using IgG to capture a ˜20 kDa protein and using a 50 kDa HF filter for TFF could facilitate permeation of the TP and low MW impurities in 7 diafiltration volumes (less than 0.2% remaining of TP and low MW impurities) while retaining more than 90% of the TP-TPBM complex. Based on the average number of amino acids present in FDA approved therapeutics 1231 and assuming an average MW of ˜120 Da for an amino acid, this TP-TPBM pair could be used on more than one third of FDA approved therapeutic proteins.

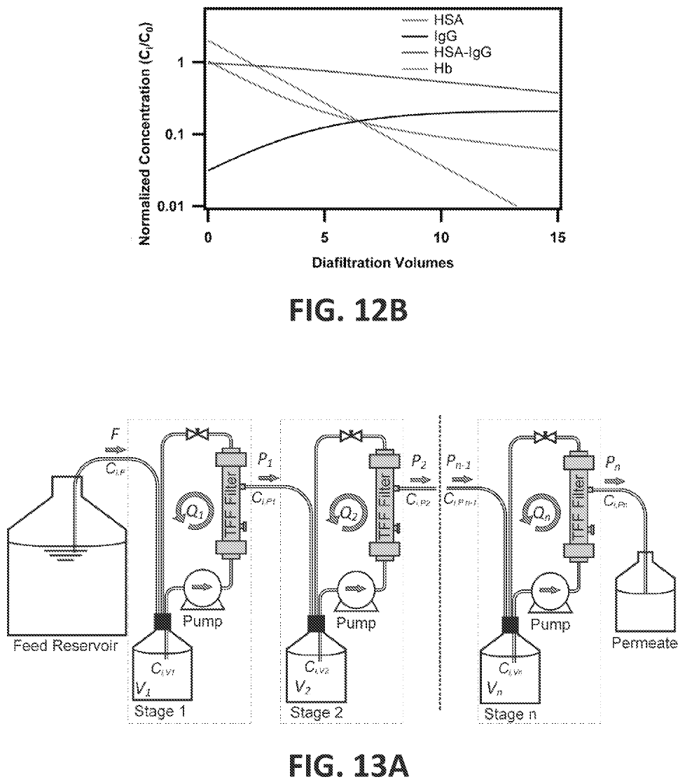

However, as demonstrated by the difference in the experimentally observed retention of the HSA-IgG pair compared to the model prediction, the model prediction would not provide accurate quantitative results unless the TP-TPBM complex had sufficiently high affinity to prevent dissociation under the conditions used during the separation. Thus, the dissociation and association reactions of the TP-TPBM pair can be added to the model by including a reaction term in Equation 2. Unfortunately, inclusion of the reaction term prevented the derivation of an analytical expression, requiring the use of an ordinary differential equations solver to find the solution. Moreover, the solution was dependent on the time to complete a diafiltration volume in addition to the reaction terms. Nonetheless, the results become more descriptive of a real system in which dissociation and association are occurring. The results using this more descriptive model on the separation of HSA from an artificial mixture of HSA and Hb is shown in FIG. 12.

As shown in FIG. 12, it was clear that the chemical reactions influenced the system performance, since the semi-log plots (FIG. 12B) would only exhibit linear concentration profiles for a purely filtration-based separation (note the Hb concentration profile). Comparing FIG. 12A to FIG. 11C shows that the chemical reactions caused a reduction in the concentration of the complex during TFF processing compared to filtration alone without accounting for the equilibrium. This additional loss of the HSA-IgG complex occurred due to complex dissociation into free IgG and HSA as the filtration process reduced the concentration of HSA in solution. Dissociation of the IgG-HSA complex is apparent due to the increase in the concentration of free IgG. Moreover, due to the dissociation of the complex, at the end of processing, the concentration profile of HSA began to asymptote to a non-zero value as the rate of protein permeation was compensated by the rate of dissociation of the complex. This final equilibrium between the protein components in the mixture agreed with the experimentally measured HPLC-SEC chromatogram. Furthermore, the model predicted that the lack of HSA permeation though the 70 kDa filter that was observed in the experiment was likely due to the protein concentrations in the permeate reaching the lower limit of detection of the HPLC system. More importantly, based on the more descriptive TFF separation model including chemical reactions, the mass-based yield of non-HSA protein components at 15 diacycles was approximately 50% of the starting value, which was close to the experimentally measured value, indicating that, by including the reaction terms, the model showed some predictive capabilities even when association and dissociation reactions were occurring.

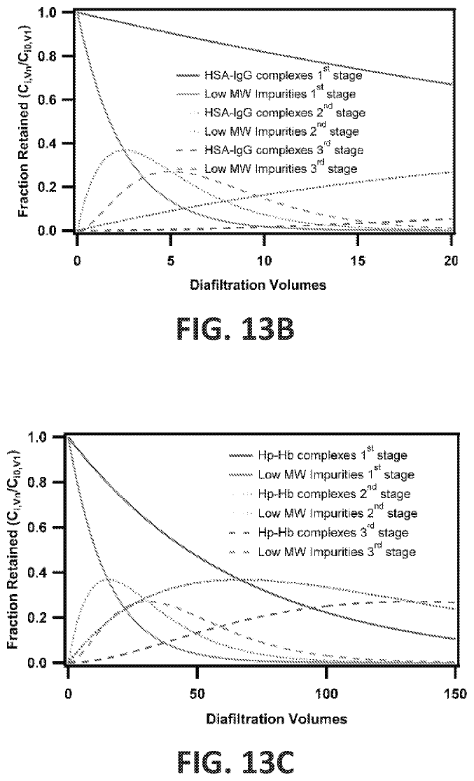

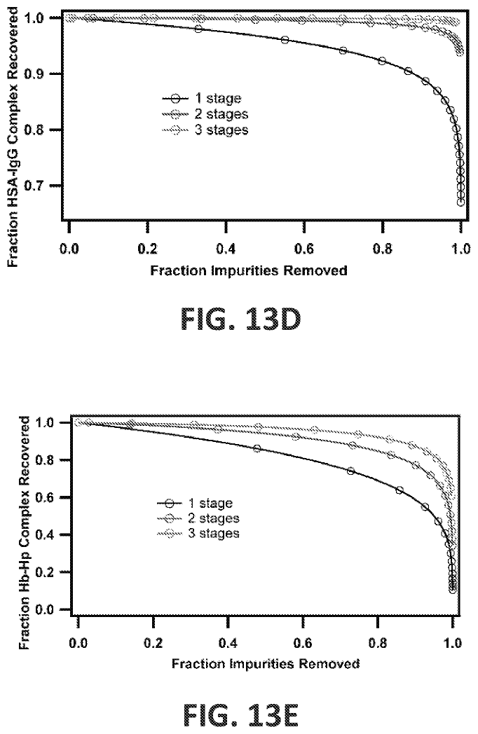

A potential drawback with the method being discussed here is the low product recovery. Yet, even though the process conditions may require many diafiltration volumes to ensure high product purity, one could implement TFF staging to increase overall product recovery without loss of purity. To demonstrate this, we used the mathematical model to demonstrate the enhanced product recovery based on a simple staged TFF setup in which the permeate of the first system is the fed to the second system and the permeate of the second system is fed to the third system as shown in FIG. 13A. To simplify the results, we used the model without chemical reactions although reactions could be added as described previously. The results of this analysis for the experimental conditions analyzed in this study (HSA-IgG and Hb-Hp complexes) for a three stage TFF system are shown in FIG. 13.

From the results shown in FIG. 13B and FIG. 13C, the first stage had the same concentration profile as in FIG. 11C and FIG. 11E, respectively. This was expected as the model in FIG. 11 was the same as a one stage system. Moreover, comparing FIGS. 13B and 13C, it was noticeable that both systems had the same overall concentration profiles. The first stage had a decay in concentration of both species while both sequential stages had an intermediate increase followed by a decay of low MW species. Furthermore, for the target complexes, the general trend was for stages 2 and 3 to increase in concentration, but at sufficiently high number of diacycles, the concentration of Hb-Hp began to decrease. More importantly, as shown in FIG. 13D and FIG. 13E, use of additional stages led to an overall increase in product recovery without loss of product purity which can be achieved over the same number of diafiltration volumes (i.e. same amount of time).

Although TFF staging has been previously shown to increase overall separation efficiency, most studies have implemented complicated configurations to increase product purity and recovery[24]. Based on our model, a simple configuration of TFF modules in series allows for improved TP-TPBM product recovery without loss of product purity. A similar approach has been used combined with affinity ultrafiltration to recover permeated TP molecules that did not bind to TPBM in the first stage[25]. Although the model can describe the overall TFF system performance, it did not consider some important factors that can influence TFF processes. Mainly, there was no effect of concentration polarization on the membrane or membrane fouling which can greatly increase protein retention within the filter during processing.[26] Moreover, we assumed that the binding of the TP to TPBM was irreversible, without consideration of protein-protein equilibria.

It is noteworthy that, with staged separation, the model predicts that the protein-protein complex may be recovered at almost any desired purity level. However, the final TP purity would be limited by the difference in retention between the TPBM and the TP on the specific HF filter used to facilitate the separation. Moreover, although in FIG. 13, the system was assumed to contain only a single impurity of MW equal to the TP, the process is robust for various MW impurities. For example, large MW impurities (i.e. larger than the TP-TPBM complex) would be primarily retained within the high MW waste during the initial filtration of the crude material whereas the TP and low MW species would permeate through the membrane. Further, any small quantities of large MW species permeated during the initial crude filtration step would later be retained during the separation of the TP from the TPBM. Furthermore, any species with MW smaller than the TP can be removed through the staging system shown in FIG. 13. Finally, any species within the size range spanning the TP to the TPBM would be the most difficult impurities to separate. However, these impurities should not be present at high levels, since the initial filtration of the crude material should retain a large fraction of these impurities.

Furthermore, these intermediate MW species could also be removed through the staging strategy shown in FIG. 13 and then partially retained during the separation of the TP from the TPBM.

Future studies could aim at using existing recombinant protein technologies to create dissociable protein complexes. For example, various tags are currently used in column affinity chromatography that could be attached to a large MW protein, forming a custom TPBM. These tagged TPBMs could lead to development of platform purification technologies using the affinity ultrafiltration approach presented here. Further, the reaction model presented could guide the design of the TPBMs binding affinity and size to ensure optimal operation of the separation process. Given that membrane filtration is already widely used for various steps in protein purification schemes, use of TFF for purification could simplify the overall production process by reducing the variety of separation equipment. Moreover, the linear scalability of TFF provides a major advantage compared to alternative purification methods as successful small-scale research systems can be easily scaled to meet market demand[27]. Further, the costs associated with this purification process may be lowered by using impure TPBM as the source of binding molecule. This could be implemented by first filtering the TP in a complex mixture over a defined MWCO membrane and using this same MWCO membrane to filter the impure TPBM. The two permeate fractions would then be mixed to yield the TP-TPBM and the low MW impurities in the mixture that could be removed by re-diafiltering through the same MWCO filter.

### Example 2: Selective Purification of Proteins Using Size-Based Separation Methods with Association and Dissociation Reactions

The assumptions for the model in Example 2 were the same as those described in the Methods Section above. Thus, for a general species i, the equation for the change in concentration within a system that includes reactions is:

$$V \frac{{dC}_{i , V}}{dt} = ( {C}_{i , IN} - {C}_{i , OUT} ) F + {i}_{} \qquad ( 6 )$$

Wherein C_{i,V} represents the concentration of species i in the system, t represents time, C_{i,IN} represents the concentration of species i being fed to the system, C_{i,OUT} represents the concentration of species i being removed from the system, and [symbol] ᵢ represents the volumetric reaction rate of species i.

Given the retention factor of species within the system (Rᵢ), C_{i,OUT} can be calculated via:

C_{i,OUT}=(1−Rᵢ)C_{i,V} (7)

Introducing the dimensionless time variable t_{D} (t_{D}=t/τ=t F/V), which is equal to the number of diafiltration volumes, the equation simplifies to:

$$\frac{{dC}_{i , V}}{{dt}_{D}} = ( {C}_{i , IN} - {C}_{i , OUT} ) + \frac{{i}_{}}{F} \qquad ( 8 )$$

For a three species system (TP, TPBM and TP-TPBM complex), the reactions occurring in the system are the following:

$$TP + TPMB {\to }^{{k}_{f}} TP - TPBM \qquad ( 9 )$$

$$TP - TPMB {\to }^{{k}_{b}} TP + TPBM \qquad ( 10 )$$

In which the dissociation constant is given by the equilibrium concentration of the individual proteins and the protein-protein complex:

$${K}_{D} = \frac{[ TP ] [ TPBM ]}{[ TP - TPBM ]} = \frac{{k}_{b}}{{k}_{f}} \qquad ( 11 )$$

Where the concentration of the species is given based on the molar concentration of the TP ([TP]), the molar concentration of the TPBM ([TPBM]) and the molar concentration of the TP-TPBM complex ([TP-TPBM]). Thus, the rate of the forward reaction is:

$${r}_{f} = {k}_{f} [ TP ] [ TPBM ] = \frac{{k}_{b}}{{K}_{D}} [ TP ] [ TPBM ] \qquad ( 12 )$$

And the rate of the reverse reaction is:

r_{b}=k_{b}[TP−TPBM]=K_{D}k_{f}[TP−TPBM] (13)

With the given equations, the volumetric rate of reaction for TP or TPBM is:

[symbol]

_{TP or TPBM}=(−r_{f}+r_{b})V=− [symbol] _{TP-TPBM} (14)

The concentrations were normalized by a reference concentration, C₀, to yield a dimensionless concentration. C′ᵢ. Further, the dissociation equilibrium constant, K_{D}, was also non-dimensionalized by C₀ yielding a dimensionless dissociation equilibrium constant K′_{D}.

Combining all these equations, the final normalized equations are:

$$\frac{{dC}_{a / b , V}^{′}}{{dt}_{D}} = [ {C}_{a / b , IN}^{′} - {C}_{a / b , V}^{′} ( 1 - {R}_{a / b} ) ] + ( - \frac{{k}_{b}}{{K}_{D}^{′}} {C}_{a}^{′} {C}_{b}^{′} + {k}_{b} {C}_{c}^{′} ) τ \qquad ( 15 )$$

$$\frac{{dC}_{c , V}^{′}}{{dt}_{D}} = [ {C}_{c , IN}^{′} - {C}_{c , V}^{′} ( 1 - {R}_{c} ) ] - ( - \frac{{k}_{b}}{{K}_{D}^{′}} {C}_{a}^{′} {C}_{b}^{′} + {k}_{b} {C}_{c}^{′} ) τ \qquad ( 16 )$$

In which species a and b are either the TP or TPBM and species c is the TP-TPBM complex.

The three differential equations corresponding to the three species (TP, TPBM and TP-TPBM) can then be simultaneous solved using an ordinary differential equation solver such as ode45 or ode23s in MATLAB.

This derivation assumes that each TPBM binds to one TP molecule to form a single TPBM complex. However, some TPBMs may bind to more than one TP, forming intermediate TP-TPBM complexes. Such a system may require equilibrium constants describing the formation of each intermediate TPBM. Moreover, these intermediate TPBM may have varying retention values that would need to be provided in the model.

This more descriptive TFF separation model with chemical reactions simplifies to the original TFF separation model with no chemical reactions, when the time for diafiltration approaches zero. This is expected, since it would indicate that the species do not have any time within the system to equilibrate during filtration. Moreover, for long diafiltration times, the system is practically always in equilibrium, which tends to be the case for fast equilibrium reactions.

Comparing Models with and without Chemical Reactions for TFF Separation of the Hb-Hp Complex

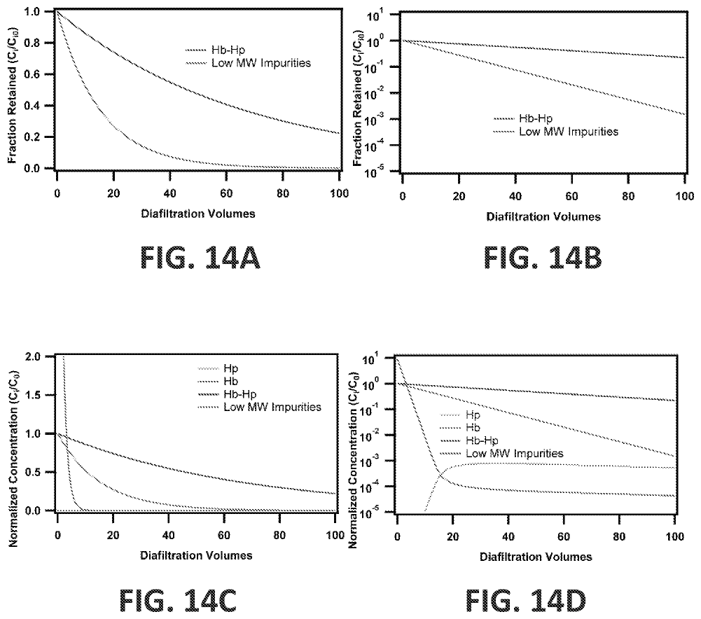

The Hp-Hb TFF separation experiment was simulated using both the TFF separation model with chemical reactions and the TFF separation model with no chemical reactions. The results are shown in FIG. 14.

As shown in FIG. 14, there was no difference in the concentration profiles of the complexed species between the two mathematical models. The excess Hb added initially to form Hb-Hp, quickly permeated out of the system. Moreover, only negligible amounts of Hp were present in the system at equilibrium and loss of Hb through the filter did not lead to appreciable dissociation of the Hb-Hp complex. Furthermore, only a negligible difference in the fraction of retained Hb-Hp was observed at 100 diafiltration volumes: from 0.226 without chemical reactions to 0.224 when chemical reactions were included.

### REFERENCES

[1] J. Romero, A. L. Zydney, Chiral separations using ultrafiltration with a stereoselective binding agent, Sep. Sci. Technol. 36 (2001) 1575-1594. https://doi.org/10.1081/SS-100103889.
[2] C. D. Filipe, R. Ghosh, Effects of protein-protein interaction in ultrafiltration based fractionation processes. Biotechnol. Bioeng. 91 (2005) 678-687. https://doi.org/10.1002/bit.20568.
[3] D. M. Kanani, R. Ghosh, C. D. M. Filipe, A novel approach for high-resolution protein-protein separation by ultrafiltration using a dual-facilitating agent, J. Memb. Sci. 243 (2004) 223-228. https://doi.org/10.1016/j.memsci.2004.06.023.
[4] A. F. Palmer, G. Sun, D. R. Harris, Tangential flow filtration of hemoglobin, Biotechnol. Prog. 25 (2009) 189-199. https://doi.org/10.1002/btpr.119.
[5] I. S. Pires, A. F. Palmer, Tangential flow filtration of haptoglobin, Biotechnol. Prog. 36 (2020) e3010. https://doi.org/10.1002/btpr.3010.
[6] C. C. Winterbourn, Oxidative reactions of hemoglobin, Methods Enzymol. 186 (1990) 265-272. https://doi.org/10.1016/0076-6879(90)86118-F.
[7] E. Chiancone, A. Alfsen, C. loppolo, P. Vecchini, A. F. Agrò, J. Wyman, E. Antonini, Studies on the reaction of haptoglobin with haemoglobin and haemoglobin chains: I. Stoichiometry and affinity. J. Mol. Biol. 34 (1968) 347-356. https://doi.org/10.1016/0022-2836(68)90258-1.
[8] E. Sada, S. Katoh. K. Sukai, M. Tohma, A. Kondo, Adsorption equilibrium in immuno-affinity chromatography with polyclonal and monoclonal antibodies, Biotechnol. Bioeng. 28 (1986) 1497-1502. https://doi.org/10.1002/bit.260281007.
[9] R Reverberi, L. Reverberi, Factors affecting the antigen-antibody reaction, Blood Transfus. 5 (2007) 227-240. https://doi.org/10.2450/2007.0047-07.
[10] W. C. Olson, T. M. Spitznagel, M. L. Yarmush, Dissociation kinetics of antigen-antibody interactions: studies on a panel of anti-albumin monoclonal antibodies, Mol. Immunol. 26 (1989) 129-136. https://doi.org/10.1016/0161-5890(89)90094-1.
[11] J. N. Herron, E. W. Voss, Analysis of heterogeneous dissociation kinetics in polyclonal populations of rabbit anti-fluorescyl-IgG antibodies, Mol. Immunol. 20 (1983) 1323-1332. https://doi.org/10.1016/0161-5890(83)90163-3.
[12] J. N. Herron, E. W. Voss. An exact procedure for the analysis of heterogeneous kinetics in polyclonal antibody populations, J. Biochem. Biophys. Methods. 8 (1983) 189-204. https://doi.org/10.1016/0165-022X(83)90067-2.
[13] F. J. Stevens, Analysis of Protein-Protein Interaction by Simulation of Small-Zone Size-Exclusion Chromatography: Application to an Antibody-Antigen Association1, 1986. https://pubs.acs.org/sharingguidelines (accessed Jan. 16, 2020).
[14] F. J. Stevens, Analysis of protein-protein interaction by simulation of small-zone size exclusion chromatography. Stochastic formulation of kinetic rate contributions to observed high-performance liquid chromatography elution characteristics, Biophys. J. 55 (1989) 1155-1167. https://doi.org/10.1016/S0006-3495(89)82912-1.
[15] Q. Li, M. Gordon. C. Cao, K. E. Ugen, D. Morgan, Improvement of a low pH antigen-antibody dissociation procedure for ELISA measurement of circulating anti-Aβ antibodies, BMC Neurosci. 8 (2007) 22. https://doi.org/10.1186/1471-2202-8-22.
[16] N. Zhang, A. F. Palmer, Liposomes surface conjugated with human hemoglobin target delivery to macrophages, Biotechnol. Bioeng. 109 (2012) 823-9. https://doi.org/10.1002/bit.24340.
[17] I. S. Pires, D. A. Belcher, R. Hickey, C. Miller, A. K. Badu-Tawiah, J. H. Baek, P. W. Buehler, A. F. Palmer, Novel manufacturing method for producing apohemoglobin and its biophysical properties, Biotechnol. Bioeng. 117 (2020) 125-145. https://doi.org/l 0.1002/bit.27193.
[18] F. Polticelli, A. Bocedi, G. Minervini, P. Ascenzi, Human haptoglobin structure and function—a molecular modelling study, FEBS J. 275 (2008) 5648-5656. https://doi.org/10.1111/j.1742-4658.2008.06690.x.
[19] P. K. Hwang, J. Greer, Interaction between hemoglobin subunits in the hemoglobin. Haptoglobin complex, J. Biol. Chem. 255 (1980) 3038-3041. http://www.ncbi.nlm.nih.gov/pubmed/7358726 (accessed Dec. 19, 2018).
[20] S. R. Wickramasinghe, S. E. Bower, Z. Chen, A. Mukherjee, S. M. Husson, Relating the pore size distribution of ultrafiltration membranes to dextran rejection, J. Memb. Sci. 340 (2009) 1-8. https://doi.org/10.1016;j.memsci.2009.04.056.
[21] K. H. Youm, W. S. Kim, Prediction of intrinsic pore properties of ultrafiltration membrane by solute rejection curves: Effects of operating conditions on pore properties, J. Chem. Eng. JAPAN. 24 (1991) 1-7. https://doi.org/10.1252/jcej.24.1.
[22] M. Waks, S. Beychok, Induced conformational states in human apohemoglobin on binding of haptoglobin 1-1. Effect of added heme as a probe of frozen structures, Biochemistry. 13 (1974) 15-22. https://doi.org/10.1021/bi00698a003.
[23] S. S. Usmani, G. Bedi, J. S. Samuel, S. Singh, S. Kalra, P. Kumar, A. A. Ahuja, M. Sharma, A. Gautam, G. P. S. Raghava, THPdb: Database of FDA-approved peptide and protein therapeutics, PLoS One. 12 (2017) e0181748. https://doi.org/10.1371/journal.pone.0181748.
[24] R. Ghosh, Novel cascade ultrafiltration configuration for continuous, high-resolution protein-protein fractionation: A simulation study, J. Memb. Sci. 226 (2003) 85-99. https://doi.org/10.1016/j.memsci.2003.08.012.
[25] J. Romero, A. L. Zydney, Staging of affinity ultrafiltration processes for chiral separations. J. Memb. Sci. 209 (2002) 107-119. https://doi.org/10.1016/50376-7388(02)00283-1.
[26] V. G. J. Rodgers, R. E. Sparks, Reduction of membrane fouling in the ultrafiltration of binary protein mixtures, AIChE J. 37 (1991) 1517-1528. https://doi.org/10.1002/aic.690371009.
[27] R. van Reis, E. M. Goodrich, C. L. Yson, L. N. Frautschy, S. Dzengeleski, H. Lutz, Linear scale ultrafiltration, Biotechnol. Bioeng. 55 (1997) 737-746. https://doi.org/10.1002/(SICI)1097-0290(19970905)55:5<737::AID-BIT4>3.0.CO;2-C.

The methods of the appended claims are not limited in scope by the specific compositions, systems, and methods described herein, which are intended as illustrations of a few aspects of the claims. Any methods that are functionally equivalent are intended to fall within the scope of the claims. Various modifications of the methods in addition to those shown and described herein are intended to fall within the scope of the appended claims. Further, while only certain representative compositions, systems, and method steps disclosed herein are specifically described, other combinations of the compositions, systems, and method steps also are intended to fall within the scope of the appended claims, even if not specifically recited. Thus, a combination of steps, elements, components, or constituents may be explicitly mentioned herein, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

1. A method for separating a target species from a solution containing additional species, the method comprising:
(i) estimating a retention factor of a target species from a molecular weight of the target species and a retention curve of a filter membrane;
(ii) calculating a number of diafiltration volumes needed to afford a desired fraction of target species based on the estimated retention factor for the target species and a net individual species molar flowrate of the target species;
(iii) filtering the solution by ultrafiltration against the filtration membrane having the retention curve from (i) and using the number of diafiltration volumes from (ii), thereby forming a fraction substantially comprising the additional species and another fraction substantially comprising the target species.

2. The method of claim 1, wherein at least 75% by weight of the target species in the solution is present in the fraction substantially comprising the target species.

3. The method of claim 1, wherein the fraction substantially comprising the target species comprises a retained fraction.

4. The method of claim 1, wherein the fraction substantially comprising the target species comprises a permeate fraction.

5. The method of claim 1, wherein the target species comprises a target protein, target protein binding molecule, target protein complex, or an impurity.

6. The method of claim 1, wherein the target species has a molecular weight of from 1 kDa to 1000 kDa, such as a molecular weight of from 1 to 250 kDa, a molecular weight of from 1 to 200 kDa, a molecular weight of from 10 to 100 kDa, or a molecular weight of from 50 kDa to 350 kDa.

7. The method of claim 1, wherein the target species comprises haptoglobin or human serum albumin.

8. The method of claim 1, wherein the estimation of the retention factor for the target species is determined from a representative retention curve generated by fitting a curve to experimentally determined or specified retention factor specifications and/or values from various sized molecules separated on a specified filter membrane.

9. The method of claim 1, wherein the estimation of the retention factor for the target species is determined from a representative retention curve generated by interpolation or extrapolation of experimentally determined retention factor specifications and/or values from various sized molecules separated on a specified filter membrane.

10. The method of claim 9, wherein the experimentally determined retention factor specifications and/or values are determined experimentally by a user who performs filtering step (iii), determined experimentally by a manufacturer of the filter membrane, provided by a manufacturer of the filter membrane, or any combination thereof.

11. The method of claim 8, wherein the representative retention curve exhibits a sigmoidal shape relating a molecule retention factor to a logarithm of molecule size.

12. The method of claim 11, wherein the molecule size comprises a molecular weight value.

13. The method of claim 1, wherein the representative retention curve exhibits a log normal distribution.

14. The method of claim 1, wherein the estimation of the retention factor for the target species is determined using the equation below:
$${R}_{i} = \frac{1}{{( \frac{b}{{MW}_{i}} )}^{n} + 1}$$
wherein b and n are regressed from experimental data for the filter membrane, Rᵢ is the retention factor for the target species i, and MWᵢ is the molecular weight of the target species.

15. The method of claim 14, wherein the molecular weight of the target species is normalized by a representative filter cut-off size that is offset by an experimentally determined value applicable to more than one analogous filter membrane.

16. The method of claim 15, wherein the more than one analogous filter membranes are filter membranes that are formed from the same materials and/or made through the same manufacturing processes, but exhibit different average pore sizes.

17. The method of claim 15, wherein the estimation of the retention factor for the target species is determined using the equation below:
$${R}_{i , j} = \frac{1}{{( \frac{{MWCO}_{j} + b}{{MW}_{i}} )}^{n} + 1}$$
wherein b and n are regressed from experimental data for a given set of analogous filter membranes, R_{i,j} is the retention factor the target species i, MWᵢ is the molecular weight of the target species, and MWCOⱼ is the molecular weight cut-off (MWCO) of the filter membrane.

18. The method according to claim 1, wherein calculating step (ii) is determined using the equation below:
$$\frac{{C}_{i , V}}{{C}_{i , {V}_{0}}} = \frac{{C}_{i , F}}{{C}_{i , {V}_{0}} ( 1 - {R}_{i} )} + ( 1 - \frac{{C}_{i , F}}{{C}_{i , {V}_{0}} ( 1 - {R}_{i} )} ) {e}^{- ( 1 - {R}_{i} ) {t}_{D}}$$
wherein Rᵢ is the retention factor of the target species estimated from step (i), C_{i,V} is the concentration of species i in a system volume, C_{i,V}_{₀} is the initial concentration of the target species i in the system volume, C_{i,F} is the concentration of the target species i in a feed stream, and t_{D} is the number of diafiltration volumes.

19. The method of claim 1, wherein the target species comprises a target protein complex, and wherein calculating step (ii) is determined using one or more of the equations below:
$$\frac{{dC}_{a / b , V}^{′}}{{dt}_{D}} = [ {C}_{a / b , IN}^{′} - {C}_{a / b , V}^{′} ( 1 - {R}_{a / b} ) ] + ( - \frac{{k}_{b}}{{K}_{D}^{′}} {C}_{a}^{′} {C}_{b}^{′} + {k}_{b} {C}_{c}^{′} ) τ \\ \frac{{dC}_{c , V}^{′}}{{dt}_{D}} = [ {C}_{c , IN}^{′} - {C}_{c , V}^{′} ( 1 - {R}_{c} ) ] - ( - \frac{{k}_{b}}{{K}_{D}^{′}} {C}_{a}^{′} {C}_{b}^{′} + {k}_{b} {C}_{c}^{′} ) τ$$
wherein a and b a target protein and a target protein binding molecule respectively and c represents the target protein complex, C_{i,V}′ is a normalized concentration of a species (a, b, or c) in a system volume, C_{i,IN}′ is a normalized concentration of a species (a, b, or c) in a feed stream, Rᵢ is the retention factor of a species (a, b, or c), τ is the time for a diafiltration volume, t_{D} is the number of diafiltration volumes, k_{b} is a dissociation rate constant for the target protein complex, and K_{D}′ is a non-dimensionalized dissociation constant for a reaction between species a, b, and c.

20. The method of claim 1, wherein the ultrafiltration of the solution is performed using tangential flow filtration.

21. The method of claim 1, wherein a fraction of the target species permeated or a fraction of the target species retained is greater than or equal to 0.90.

22. The method of claim 1, wherein a fraction of the target species permeated or a fraction of the target species retained is less than or equal to 0.10.

23. The method of claim 1, wherein the additional species comprise an impurity.

24. The method of claim 1, wherein the method comprises:
(i) estimating the retention factor of the target species from the molecular weight of the target protein or target protein complex and the retention curve of the filter
(ii) estimating the retention factor for the impurity from the molecular weight of the impurity and the retention curve of the filter;
(iii) calculating a number of diafiltration volumes needed to afford a desired fraction of target species and a desired fraction of impurity based on the retention factor of the target species, the retention factor of the impurity, and a net individual species molar flowrate for the target species and impurity;
(iv) filtering the solution by ultrafiltration against the filtration membrane having the retention curve from steps (i) and (ii) using the number of diafiltration volumes from step (iii), thereby forming a fraction substantially comprising the impurity and another fraction substantially comprising the target species.

25. The method of claim 24, wherein the fraction substantially comprising the target species comprises a retained fraction and the fraction substantially comprising the impurity comprises a permeate fraction.

26. The method of claim 25, wherein a fraction of the target species retained is greater than or equal to 0.90, and wherein a fraction of the impurity retained is less than or equal to 0.10.

27. The method of claim 26, further comprising increasing the number of diafiltration volumes to increase the fraction of the target species retained, decrease the fraction of the impurity retained, or a combination thereof.

28. The method of claim 26, further comprising selecting a filter membrane having a molecular weight cut-off effective to increase the fraction of the target species retained, decrease the fraction of the impurity retained, or a combination thereof.

29. The method of claim 24, wherein the fraction substantially comprising the target species comprises a permeate fraction and the fraction substantially comprising the impurity comprises a retained fraction.

30. The method of claim 29, wherein a fraction of the target species permeated is greater than or equal to 0.90, and wherein a fraction of the impurity permeated is less than or equal to 0.10.

31. The method of claim 30, further comprising increasing the number of diafiltration volumes to increase the fraction of the target species permeated, decrease the fraction of the impurity permeated, or a combination thereof.

32. The method of claim 30, further comprising selecting a filter membrane having a molecular weight cut-off effective to increase the fraction of the target species permeated, decrease the fraction of the impurity permeated, or a combination thereof.

33. A method for separating a target protein from a sample solution containing a plurality of impurities, the method comprising:
(i) estimating the retention factor of the target protein from the molecular weight of the target protein and the retention curve of a first filtration membrane having a first molecular weight cut-off value;
(ii) estimating the retention factor of a first impurity from the molecular weight of the first impurity and the retention curve of the first filtration membrane having the first molecular weight cut-off value;
(iii) calculating a first number of diafiltration volumes needed to afford a desired fraction permeated for the target protein based on the retention factor of the target protein from step (i) and a net molar flowrate for the target protein, and a desired fraction retained for the first impurity based on the retention factor of first impurity from step (ii) and a net molar flowrate for the first impurity;
(iv) filtering the solution by ultrafiltration against the first filtration membrane using the first number of diafiltration volumes from step (iii), thereby forming a first retentate fraction substantially comprising the first impurity and a first permeate fraction substantially comprising the target protein;
(v) contacting the first permeate fraction with a binding molecule that selectively associates with the target protein to form a target protein complex having a molecular weight above the first molecular weight cut-off value of the first membrane;
(vi) estimating a retention factor for the target protein complex from the molecular weight of the target protein complex and the retention curve for a second filter membrane having a second molecular weight cut-off value;
(vii) estimating a retention factor for a second impurity present in the first permeate fraction from the molecular weight of the second impurity and the retention curve for a second filter membrane having a second molecular weight cut-off value;
(viii) calculating a second number of diafiltration volumes needed to afford a desired fraction retained for the target protein complex based on the retention factor of the target protein complex from step (vi) and a net molar flowrate for the target protein complex, and a desired fraction permeated for the second impurity based on the retention factor of second impurity from step (vii) and a net molar flowrate for the second impurity; and
(ix) filtering the first permeate fraction by ultrafiltration against the second filtration membrane using the second number of diafiltration volumes from step (viii), thereby forming a second retentate fraction substantially comprising the target protein complex and a second permeate fraction substantially comprising the second impurity.

34-40. (canceled)

41. The method of claim 1, wherein the method is a continuous process, and wherein the calculating steps account for changes in species concentrations over time.
